(19) United States
(12) Patent Application Publication
(10) Pub. No.: US 2026/0262987 A1
(43) Pub. Date: Sep. 10, 2026

(54) BIOELECTRIC POTENTIAL MEASUREMENT DEVICE

(71) Applicants: SEIKO GROUP CORPORATION, Tokyo (JP); The University of Osaka, Suita-shi, Osaka (JP)

(72) Inventors: Mikishige SUGASA, Matsudo-shi (JP); Yoshiyuki KAIHO, Matsudo-shi (JP); Kazumasa SASAHARA, Matsudo-shi (JP); Teppei ARAKI, Suita-shi (JP); Tsuyoshi SEKITANI, Suita-shi (JP)

(21) Appl. No.: 19/167,683

(22) PCT Filed: Mar. 8, 2024
(86) PCT No.: PCT/JP2024/009082

§ 371 Date: Sep. 22, 2025

(30) Foreign Application Priority Data
Mar. 24, 2023 (JP) 2023-048423

(51) Int. Cl. A61B 5/257

(52) U.S. Cl. CPC A61B 5/257

(57) ABSTRACT

A bioelectric potential measurement device includes an electrode sheet configured to acquire a biological signal, a device which has a contact portion to be connected to the electrode sheet and a connection member configured to connect the electrode sheet to the contact portion by holding the electrode sheet between the device and the connection member, in which the connection member has a fitting portion which fits into the device, and the electrode sheet has a shape corresponding to the fitting portion.

### RELATED APPLICATIONS

This Application is a 371 application of PCT/JP2024/009082 having an international filing date of Mar. 8, 2024, which claims priority to Japanese Patent Application No. 2023-048423, filed on Mar. 24, 2023, the content of each of which is incorporated herein by reference.

### TECHNICAL FIELD

The present invention relates to a bioelectric potential measurement device.

### BACKGROUND ART

Patent Document 1 discloses a biological information output device which is worn on skin of a subject, detects an electrical biological signal generated in a body of the subject from the skin, and outputs biological information obtained by processing the biological signal.

### CITATION LIST

### Patent Document

Patent Document 1: Japanese Unexamined Patent Application, First Publication No. 2022-120573

### SUMMARY OF INVENTION

### Technical Problem

In the above-described biological information output device (bioelectric potential measurement device), a housing (device) and an attachment sheet (electrode sheet) are connected by a substantially C-shaped housing holder (connection member). Therefore, in the related art, it is necessary to increase the sizes of the housing and the housing holder according to a size of the attachment sheet.

The present invention has been made in view of the above-described problem, and an object of the present invention is to provide a bioelectric potential measurement device in which an electrode sheet can be reliably connected to a device without being affected by a size of the electrode sheet.

### Solution to Problem

(1): A bioelectric potential measurement device according to one aspect of the present invention includes an electrode sheet configured to acquire a biological signal, a device which has at least one contact portion to be connected to the electrode sheet, and a connection member configured to connect the electrode sheet to the at least one contact portion by holding the electrode sheet between the device and the connection member, in which the connection member has at least one fitting portion which fits into the device, and the electrode sheet has a shape corresponding to the at least one fitting portion.

With the bioelectric potential measurement device according to the present aspect, the electrode sheet can be reliably connected to the contact portion of the device by holding the electrode sheet between the device and the connection member. In addition, since the electrode sheet has a shape corresponding to the fitting portion of the connection member, it is not necessary to increase the sizes of the device and the connection member in accordance with the size of the electrode sheet.

Therefore, with the bioelectric potential measurement device according to the present aspect, it is possible to obtain a bioelectric potential measurement device in which the electrode sheet can be reliably connected to the device without being affected by the size of the electrode sheet.
(2): In the bioelectric potential measurement device according to the aspect of (1), the device may have a fitting target portion into which the at least one fitting portion to be fitted.

In this case, the connection member is less likely to come off the device.
(3): In the bioelectric potential measurement device according to the aspect of (1) or (2), the at least one fitting portion comprises a plurality of fitting portions, and the plurality of the fitting portions may be provided in a pair in at least a lateral direction of the electrode sheet.

In this case, the device can be reduced in size in a longitudinal direction of the electrode sheet.
(4): In the bioelectric potential measurement device according to the aspect of (3), the plurality of the fitting portions may have a first fitting portion which is provided in a pair in the lateral direction, and a second fitting portion which fits into the device at a position different from the first fitting portion.

In this case, the connection member is even less likely to come off the device.
(5): In the bioelectric potential measurement device according to the aspect of (4), the second fitting portion may be provided in parallel with the first fitting portion.

In this case, stability of the fitting of the connection member can be increased.
(6): In the bioelectric potential measurement device according to the aspect of (4) or (5), the second fitting portion may fit into the device from an orientation different from the first fitting portion.

In this case, rotation of the device around an axis extending in the lateral direction of the electrode sheet can be restricted by the fitting of the second fitting portion.
(7): In the bioelectric potential measurement device according to any one of the aspects of (4) to (6), the second fitting portion may slide in a longitudinal direction of the electrode sheet to fit into the device.

In this case, the second fitting portion is easily fitted to the device.
(8): In the bioelectric potential measurement device according to any one of the aspects of (1) to (7), the at least one contact portion comprises a plurality of contact portions, and the connection member may connect the electrode sheet to the plurality of the contact portions.

In this case, the electrode sheet can be simultaneously connected to the plurality of the contact portions.
(9): In the bioelectric potential measurement device according to any one of the aspects of (1) to (8), a positioning mechanism which positions the electrode sheet and the at least one contact portion may be provided, and the positioning mechanism may include at least one the fitting portion.

In this case, since the fitting portion also serves as the positioning mechanism, the number of components of the bioelectric potential measurement device can be reduced.
(10) In the bioelectric potential measurement device according to the aspect of (9), the positioning mechanism may include at least one through-hole which is formed in the electrode sheet corresponding to the at least one fitting portion and through which the at least one fitting portion is disposed.

In this case, since the fitting portion of the connection member is inserted through the through-hole formed in the electrode sheet and fits into the device, an outer shape of the electrode sheet can be freely expanded.
(11): In the bioelectric potential measurement device according to the aspect of (10), the at least one through-hole comprises a plurality of through-holes, and the plurality of the through-holes may be provided in a pair, and the at least one contact portion may be disposed between the pair of the through-holes in a plan view.

In this case, since a pair of the fitting portions of the connection member is inserted into the pair of the through-holes formed in the electrode sheet, the electrode sheet can be positioned with high accuracy with respect to the contact portion.
(12): In the bioelectric potential measurement device according to the aspect of (9), the positioning mechanism may include a constricted portion which is formed on an outer edge of the electrode sheet corresponding to the at least one fitting portion and on which the at least one fitting portion is disposed.

In this case, even in a case where the through-hole cannot be formed in the electrode sheet, the electrode sheet can be positioned with respect to the contact portion by the constricted portion.
(13) In the bioelectric potential measurement device according to any one of the aspects of (1) to (12), the connection member may include an elastic portion formed at a facing portion, the facing portion faces the at least one contact portion with the electrode sheet therebetween.

In this case, the electrode sheet can be reliably connected to the contact portion by pressing the electrode sheet with the elastic portion.
(14): In the bioelectric potential measurement device according to any one of the aspects of (1) to (13), the connection member may include a projected portion formed at a facing portion and protrudes toward the electrode sheet, the facing portion faces the at least one contact portion with the electrode sheet therebetween.

In this case, the electrode sheet can be reliably connected to the contact portion by pressing the electrode sheet with the projected portion.
(15): In the bioelectric potential measurement device according to any one of the aspects of (1) to (13), the connection member may include a recessed portion formed at a facing portion and recessed toward a side opposite to the electrode sheet, the facing portion faces the at least one contact portion with the electrode sheet therebetween.

In this case, the recessed portion can suppress the electrode sheet from being pressed against the contact portion with an excessive force.
(16): In the bioelectric potential measurement device according to any one of the aspects of (1) to (15), the connection member may include a facing portion, the facing portion faces the at least one contact portion with the electrode sheet therebetween, and the facing portion may be transparent.

In this case, connection status between the electrode sheet and the contact portion can be visualized.
(17): In the bioelectric potential measurement device according to any one of the aspects of (1) to (16), the connection member may include an extending portion which extends laterally from a side edge surface of the electrode sheet in a lateral direction of the electrode sheet.

In this case, the bioelectric potential measurement device is easily detachable from the living body by hooking a finger on the extending portion.
(18): In the bioelectric potential measurement device according to any one of the aspects of (1) to (16), the connection member may be disposed inside an outer edge of the electrode sheet in a plan view.

In this case, since the connection member is covered with the electrode sheet, it is difficult for a finger or the like to be caught on the connection member, so that the bioelectric potential measurement device is unlikely to be unintentionally peeled off from the living body.
(19): In the bioelectric potential measurement device according to any one of the aspects of (1) to (18), the connection member may configure to be movable and integrated with the device.

In this case, since the device and the connection member are integrated, the connection member can be prevented from being lost. In addition, connection stability between the device and the connection member can be improved.
(20): In the bioelectric potential measurement device according to any one of the aspects of (1) to (19), the electrode sheet may have a transparent electrode.

In this case, visibility is improved by providing the transparent electrode as the electrode sheet, and thus misregistration is easily confirmed.
(21): In the bioelectric potential measurement device according to any one of the aspects of (1) to (20), at least one of the device or the connection member may be provided with an abutting portion which abuts the other through a portion of the electrode sheet which does not overlap with the at least one contact portion.

In this case, the abutting portion can serve as a spacer so that an excessive force is not applied to the contact portion.
(22): In the bioelectric potential measurement device according to the aspect of (21), the abutting portion may have a curved corner portion.

In this case, it is possible to reduce load (stress concentration) applied to the electrode sheet from the corner portion of the abutting portion.
(23): In the bioelectric potential measurement device according to any one of the aspects of (1) to (22), the at least one contact portion may have a curved corner portion.

In this case, it is possible to reduce load (stress concentration) applied to the electrode sheet from the corner portion of the contact portion.
(24): In the bioelectric potential measurement device according to any one of the aspects of (1) to (23), the electrode sheet may have at least one conductive portion to be connected to the at least one contact portion, and the at least one conductive portion may include a terminal portion which is in contact with the at least one contact portion, an electrode portion which is in contact with a biological side, and a wiring portion configured to connect the terminal portion and the electrode portion to each other.

In this case, the electrode portion in contact with the biological side can be disposed at a position away from the contact portion on the device side.
(25): In the bioelectric potential measurement device according to the aspect of (24), the at least one conductive portion may comprise a plurality of conductive portions having a plurality of the terminal portions and a plurality of the electrode portions, and a terminal group including the plurality of the terminal portions and an electrode group including the plurality of the electrode portions may be arranged to be spaced from each other in a plan view.

In this case, since the electrode group to be connected to the biological side is disposed to be separated from the terminal group to be connected to the device side by the connection member, it is possible to suppress peeling of the electrode group from the biological side.
(26): In the bioelectric potential measurement device according to the aspect of (25), the plurality of the electrode portions may be linearly arranged in the electrode group, and the terminal group may be disposed on an extension line of the plurality of the electrode portions.

In this case, the electrode sheet can be reduced in size.
(27): In the bioelectric potential measurement device according to any one of the aspects of (24) to (26), a surface of the terminal portion may be harder than the wiring portion.

In this case, since the surface of the terminal portion is hardened, the terminal portion can be reliably connected to the contact portion, so that the connection between the terminal portion and the contact portion can be stabilized.
(28): In the bioelectric potential measurement device according to any one of the aspects of (24) to (26), a surface of the terminal portion may have a hardness equal to or lower than the hardness of the wiring portion.

In this case, since the surface of the terminal portion is softened, the terminal portion can be deformed to follow the contact portion, so that the connection between the terminal portion and the contact portion can be stabilized.
(29): In the bioelectric potential measurement device according to any one of the aspects of (24) to (28), the device may have an accommodation portion in which the connection member is accommodated, and the contact portion to which the terminal portion is connected may be disposed in the accommodation portion.

In this case, since the connection member does not protrude to the biological side, it is possible to reduce discomfort on the biological side.
(30): In the bioelectric potential measurement device according to the aspect of (29), the accommodation portion may have a side wall portion on which the contact portion is disposed, the electrode sheet may have a bent portion which is held between the side wall portion and the at least one fitting portion in the accommodation portion, and the terminal portion to be connected to the contact portion may be provided in the bent portion.

In this case, since the connection member does not need to be disposed on the biological side with respect to the electrode sheet, peeling of the electrode sheet from the biological side can be suppressed.
(31): In the bioelectric potential measurement device according to any one of the aspects of (24) to (30), the at least one fitting portion may have a restricting portion which restricts deformation of the electrode sheet in a first direction.

In this case, since stretch and contraction of the electrode sheet in the first direction is restricted, the connection between the terminal portion and the contact portion can be stabilized.
(32): In the bioelectric potential measurement device according to the aspect of (31), the terminal portion may be formed to be long in a second direction intersecting with the first direction.

In this case, by extending the terminal portion in the second direction in which the stretch and contraction of the electrode sheet is not restricted, the connection between the terminal portion and the contact portion can be stabilized.
(33): In the bioelectric potential measurement device according to the aspect of (31) or (32), the electrode portions may be provided separately in a second direction intersecting with the first direction.

In this case, in a case where the electrode sheet stretches and contracts in the second direction, it is difficult for stress to be applied to the electrode portion.
(34): In the bioelectric potential measurement device according to any one of the aspects of (31) to (33), the connection member may include a facing portion, the facing portion faces the at least one contact portion with the electrode sheet therebetween, and the electrode portion may be disposed at a distance from the connection member in a second direction intersecting with the first direction, the distance being larger than the thickness of the facing portion.

In this case, the peeling of the electrode portion due to influence of floating of the electrode sheet from the biological side, caused by the thickness of the facing portion, can be suppressed.
(35): In the bioelectric potential measurement device according to any one of the aspects of (1) to (34), the electrode sheet may include, in a vicinity of the at least one contact portion, a shape holding portion which is harder than a base material of the electrode sheet.

In this case, it is possible to stabilize positioning of the electrode sheet and the contact portion.
(36): In the bioelectric potential measurement device according to any one of the aspects of (1) to (35), in which the connection member may include an adhesive portion which is in pressure-sensitive adhesive contact with a biological side on a surface facing a side opposite to the at least one contact portion.

In this case, since the connection member is pressure-bonded to the biological side, it is possible to suppress the electrode sheet from being peeled off from the biological side with the connection member as a starting point.
(37): In the bioelectric potential measurement device according to any one of the aspects of (1) to (36), in which the connection member may include a connection member-side electrode which is in contact with a biological side on a surface facing a side opposite to the contact portion, and the device may include a second contact portion to be connected to the connection member-side electrode.

In this case, since the electrode can be disposed at a position overlapping with the facing portion of the connection member in a plan view, the degree of freedom in the disposition of the electrode can be improved.
(38): In the bioelectric potential measurement device according to any one of the aspects of (1) to (37), the connection member may be integrated with the electrode sheet.

In this case, assembly of the bioelectric potential measurement device is facilitated.
(39): In the bioelectric potential measurement device according to any one of the aspects of (1) to (38), in which the device may have an attachment and detachment mechanism which is attachable to and detachable from the connection member.

In this case, it is possible to suppress damage to the connection member in a case of detaching the electrode sheet from the device.
(40): In the bioelectric potential measurement device according to the aspect of (39), in which the attachment and detachment mechanism may include a movement member which is movable between a fitting position where the fitting portion fits and a non-fitting position where the fitting portion is disengaged from the fitting position, and a biasing member which biases the movement member from the non-fitting position toward the fitting position.

In this case, the connection member can be removed from the device by moving the movement member fitted to the fitting portion against biasing of the biasing member.
(41): In the bioelectric potential measurement device according to the aspect of (39) or (40), in which the attachment and detachment mechanism may include a button portion which is displaced in response to attachment and detachment of the connection member.

In this case, the connection member can be detached from the device by displacing the button portion.

### Advantageous Effects of Invention

According to the aspects of the present invention, it is possible to provide a bioelectric potential measurement device in which the electrode sheet can be reliably connected to the device without being affected by the size of the electrode sheet.

### BRIEF DESCRIPTION OF DRAWINGS

FIG. 1 is a view representing an example of use of a bioelectric potential measurement device according to a first embodiment.

FIG. 2 is an exploded perspective view of the bioelectric potential measurement device according to the first embodiment.

FIG. 3 is across-sectional view of the bioelectric potential measurement device according to the first embodiment along a lateral direction.

FIG. 4 is a perspective view of a connection member according to a second embodiment.

FIG. 5 is a perspective view of a connection member according to a third embodiment.

FIG. 6 is a perspective view of a connection member according to a fourth embodiment.

FIG. 7 is a perspective view of a bioelectric potential measurement device according to a fifth embodiment.

FIG. 8 is a perspective view of a bioelectric potential measurement device according to a sixth embodiment.

FIG. 9 is a cross-sectional view of a main part of the bioelectric potential measurement device according to the sixth embodiment.

FIG. 10 is a cross-sectional view of a main part of a bioelectrical potential measurement device according to a seventh embodiment.

FIGS. 11A and 11B are simplified views showing a bioelectric potential measurement device according to an eighth embodiment.

FIG. 12 is a schematic view of a bioelectrical potential measurement device according to a ninth embodiment.

FIG. 13 is a plan view of an electrode sheet according to a tenth embodiment.

FIG. 14 is a plan view of an electrode sheet according to an eleventh embodiment.

FIG. 15 is a plan view of an electrode sheet according to a comparative example of the eleventh embodiment.

FIG. 16 is a perspective view of a connection member according to a twelfth embodiment.

FIG. 17 is a perspective view of a connection member according to a thirteenth embodiment.

FIG. 18 is a schematic cross-sectional view of a bioelectric potential measurement device according to a fourteenth embodiment along a longitudinal direction.

FIG. 19 is a plan view of an electrode sheet according to a fifteenth embodiment.

FIG. 20 is a view representing an example of use of a bioelectric potential measurement device according to a sixteenth embodiment.

FIG. 21 is a schematic cross-sectional view of a bioelectric potential measurement device according to a seventeenth embodiment along a longitudinal direction.

FIG. 22 is a schematic cross-sectional view taken along a longitudinal direction of an electrode sheet according to an eighteenth embodiment.

FIG. 23 is a schematic cross-sectional view taken along a longitudinal direction of an electrode sheet according to a nineteenth embodiment.

FIG. 24 is a schematic view of a bioelectrical potential measurement device according to a twentieth embodiment.

FIG. 25 is an exploded perspective view of a bioelectric potential measurement device according to a twenty-first embodiment.

FIG. 26 is a schematic cross-sectional view of a bioelectric potential measurement device according to a twenty-second embodiment along a longitudinal direction.

FIG. 27 is a schematic cross-sectional view of a bioelectric potential measurement device according to a twenty-third embodiment along a longitudinal direction.

FIG. 28 is a schematic cross-sectional view of a bioelectric potential measurement device according to a twenty-fourth embodiment along a longitudinal direction.

FIG. 29 is a schematic cross-sectional view of a bioelectric potential measurement device according to a twenty-fifth embodiment along a longitudinal direction.

FIG. 30 is an exploded view of the bioelectric potential measurement device shown in FIG. 29.

FIG. 31 is a plan view of an electrode sheet according to a twenty-sixth embodiment.

FIG. 32 is a plan view of an electrode sheet according to a twenty-seventh embodiment.

FIG. 33 is a plan view of an electrode sheet according to a twenty-eighth embodiment.

FIG. 34 is an exploded perspective view of a bioelectric potential measurement device according to a twenty-ninth embodiment.

### DESCRIPTION OF EMBODIMENTS

Hereinafter, embodiments according to the present invention will be described with reference to the drawings.

### First Embodiment

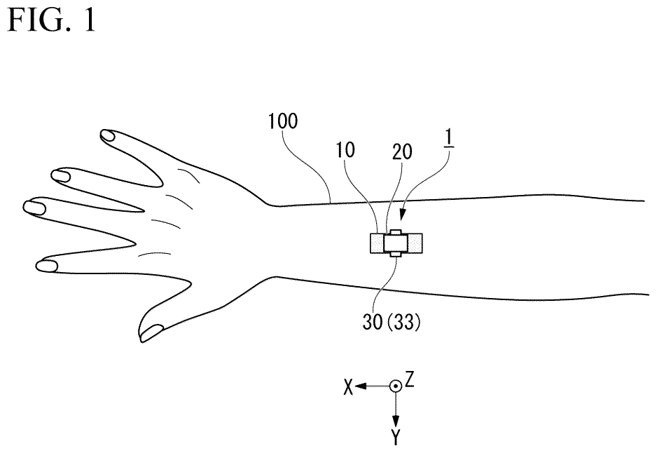

FIG. 1 is a view representing an example of use of a bioelectric potential measurement device 1 according to a first embodiment.

The bioelectric potential measurement device 1 is worn on a living body 100, and measures a biological signal of the living body 100. In the example shown in FIG. 1, the bioelectric potential measurement device 1 is worn on an arm portion of the living body 100, and measures a myogenic potential generated in a case where a muscle cell contracts through a skin of the arm portion. The bioelectric potential measurement device 1 may measure, for example, a cardiogram as a biological signal other than the myogenic potential.

The bioelectric potential measurement device 1 includes an electrode sheet 10 configured to acquire a biological signal, a device 20 which is connected to the electrode sheet 10, and a connection member 30 configured to connect the electrode sheet 10 to the device 20. The electrode sheet 10 is formed in a substantially rectangular shape in a plan view. A skin-side surface of the electrode sheet 10 is a pressure-sensitive adhesive surface, and thus the electrode sheet 10 can maintain the attached state even during exercise. In addition, the bioelectric potential measurement device 1 is small and light in order to provide a low attachment feeling.

In addition, in the following description, the XYZ Cartesian coordinate system may be set, and a positional relationship of respective members may be described with reference to the XYZ Cartesian coordinate system. The X-axis direction is set to a longitudinal direction of the electrode sheet 10. The Y-axis direction is set to a lateral direction of the electrode sheet 10. The Z-axis direction is set to a thickness direction of the electrode sheet 10.

Hereinafter, for convenience of description, the device 20 side with respect to the electrode sheet 10 may be referred to as an upper side (+Z side); and a side opposite to the device 20 with respect to the electrode sheet 10 may be referred to as a lower side (−Z side). The +Z side may not be the upper side in the direction of gravity.

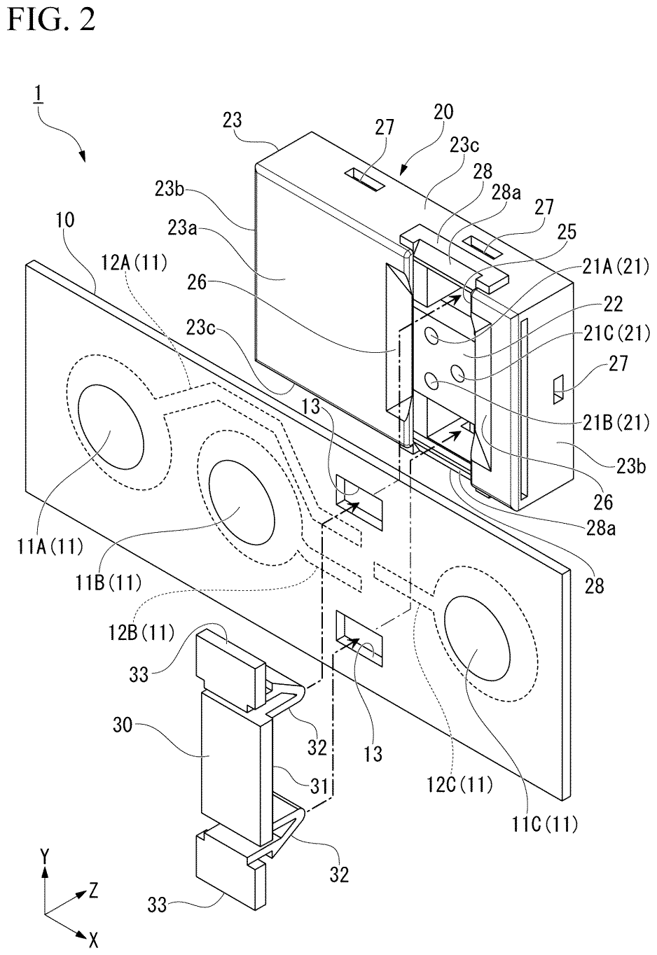

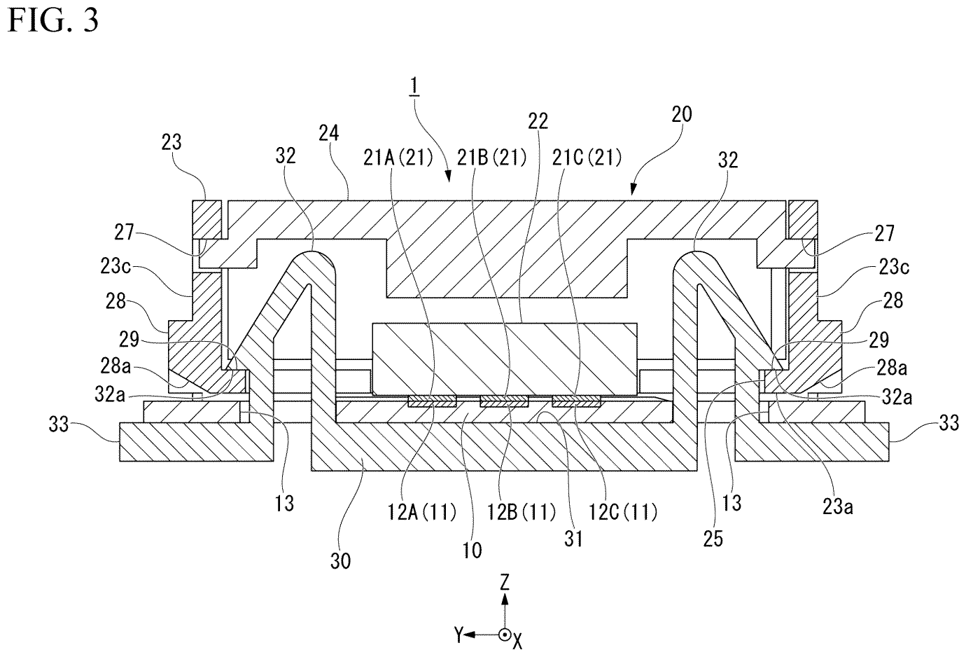

FIG. 2 is an exploded perspective view of the bioelectric potential measurement device 1 according to the first embodiment. FIG. 3 is a cross-sectional view of the bioelectric potential measurement device 1 according to the first embodiment along the lateral direction.

As shown in these drawings, the bioelectric potential measurement device 1 has a configuration in which the electrode sheet 10 is held between the device 20 and the connection member 30.

The electrode sheet 10 is, for example, a flexible printed wiring board, and has a sheet-shaped base material which is elastically deformable and has electrical insulating properties. The base material of the electrode sheet 10 is formed of, for example, polyimide, urethane, or the like. The electrode sheet 10 includes a plurality of conductive portions 11. The conductive portion 11 may be formed of a transparent electrode.

The plurality of conductive portions 11 includes a first electrode portion 11A to a third electrode portion 11C, and a first wiring portion 12A to a third wiring portion 12C. The first electrode portion 11A, the second electrode portion 11B, and the third electrode portion 11C are formed in a circular shape in a plan view as viewed from the Z-axis direction. The first electrode portion 11A, the second electrode portion 11B, and the third electrode portion 11C are provided in a row at intervals in the longitudinal direction (X-axis direction) of the electrode sheet 10.

The first electrode portion 11A, the second electrode portion 11B, and the third electrode portion 11C are exposed to the lower surface side (−Z side) of the electrode sheet 10, and come into contact with the living body 100. The first electrode portion 11A, the second electrode portion 11B, and the third electrode portion 11C may be a dry electrode or a wet electrode. In a case of a wet electrode, the first electrode portion 11A, the second electrode portion 11B, and the third electrode portion 11C come into contact with the skin in a state in which a medium such as gel is interposed therebetween.

The first wiring portion 12A, the second wiring portion 12B, and the third wiring portion 12C are formed on the upper surface side (+Z side) of the electrode sheet 10. The first wiring portion 12A is connected to the first electrode portion 11A. The second wiring portion 12B is connected to the second electrode portion 11B. The third wiring portion 12C is connected to the third electrode portion 11C.

The electrode sheet 10 has a shape corresponding to a fitting portion 32 (described later) of the connection member 30. Specifically, a through-hole 13 is formed in the electrode sheet 10, through which the fitting portion 32 is disposed. The through-hole 13 is formed in a pair spaced apart in the lateral direction. The through-hole 13 is formed in a slit shape extending in the X-axis direction.

End parts of the first wiring portion 12A, the second wiring portion 12B, and the third wiring portion 12C extend to a space between the pair of the through-holes 13. The end parts of the first wiring portion 12A, the second wiring portion 12B, and the third wiring portion 12C are arranged at intervals in the Y-axis direction between the pair of the through-holes 13, and are alternately arranged in the X-axis direction. Specifically, the end part of the third wiring portion 12C is disposed on the +X side with respect to the end parts of the first wiring portion 12A and the second wiring portion 12B. In this manner, it is possible to prevent erroneous attachment due to different orientation of the electrode sheet 10. It is sufficient that the end parts of the first wiring portion 12A, the second wiring portion 12B, and the third wiring portion 12C are disposed at positions corresponding to three contact portions 21 described later.

The device 20 has a contact portion 21 connected to the electrode sheet 10. The device 20 includes a substrate 22 on which the contact portion 21 is formed, a device case 23 which accommodates the substrate 22, and a device cover 24 (see FIG. 3) which covers the device case 23. The contact portion 21 protrudes downward (−Z side) from a lower surface of the substrate 22. As a result, the contact portion 21 is easily connected to the electrode sheet 10 which is soft (easily escapes from pressing). The contact portion 21 has a dome shape in which solder or the like is provided at each terminal. In addition, the height (amount of protrusion with respect to the substrate 22) of the contact portion 21 is, for example, approximately 0.15 mm±0.05 mm.

As shown in FIG. 2, three (first contact portion 21A to third contact portion 21C) contact portions 21 are provided in response to the number and the arrangement of the end parts of the first wiring portion 12A, the second wiring portion 12B, and the third wiring portion 12C. Specifically, the first contact portion 21A is connected to the end part of the first wiring portion 12A. The second contact portion 21B is connected to the end part of the second wiring portion 12B. The third contact portion 21C is connected to the end part of the third wiring portion 12C. The end part of the wiring portion may be larger than the contact portion 21 on the X-Y plane. As a result, even in a case where the device 20 is small, it is easy to deal with the misregistration of the electrode sheet 10.

The device 20 measures the myogenic potential from a potential difference measured by two electrode portions among the first electrode portion 11A to the third electrode portion 11C through the first contact portion 21A to the third contact portion 21C. In addition, the device 20 removes noise included in the myogenic potential with a potential measured in the remaining one electrode portion among the first electrode portion 11A to the third electrode portion 11C, as a reference. Specifically, in a case where the first electrode portion 11A and the second electrode portion 11B are used as measurement electrodes and the third electrode portion 11C is used as a reference electrode, a first differential signal which is a difference between signals of the first electrode portion 11A and the third electrode portion 11C, and a second differential signal which is a difference between signals of the second electrode portion 11B and the third electrode portion 11C are first calculated. Next, a difference between the first differential signal and the second differential signal is calculated. As a result, components other than the target myogenic potential can be removed. As another method, a signal component common to the first electrode portion 11A and the second electrode portion 11B is calculated, and a waveform of a signal having an opposite phase to the signal is applied from the third electrode portion 11C to the skin to remove noise from the signal measured by the first electrode portion 11A and the second electrode portion 11B. As a result, the myogenic potential obtained by the difference between the first electrode portion 11A and the second electrode portion 11B can also be measured in a state in which the noise is removed.

The above-described processing is executed based on a program stored in advance by a central processing unit (CPU), a memory, an input/output circuit, an IC chip, and other electronic components, which are provided on the substrate 22. Although not shown, the device 20 includes a communication device which performs wireless communication with an external device, and a power supply unit which supplies power to each electronic component.

The device case 23 is, for example, a resin-molded part, and is formed in a rectangular box shape as shown in FIG. 2. The upper side (+Z) of the device case 23 is open, and the opening is covered with the device cover 24 (see FIG. 3). As shown in FIG. 2, the device case 23 includes a bottom surface 23a facing the lower side (−Z side), a pair of side wall surfaces 23b facing the longitudinal direction (X-axis direction), and a pair of side wall surfaces 23c facing the lateral direction (Y-axis direction).

An opening portion 25 extending in the lateral direction (Y-axis direction) is formed in the bottom surface 23a of the device case 23. A part of the substrate 22 accommodated in the device case 23, and the first contact portion 21A to the third contact portion 21C are exposed from the opening portion 25. The opening portion 25 forms a gap into which the fitting portion 32 of the connection member 30 can be inserted on both sides of the substrate 22 in the lateral direction (Y-axis direction).

On the bottom surface 23a of the device case 23, inclined portions 26 in which corners of a part of the opening edge are rounded are formed on the +X side and the −X side of the opening portion 25. The inclined portion 26 reduces load (stress concentration) applied to the electrode sheet 10 from the opening edge of the opening portion 25 in a case where the electrode sheet 10, the device 20, and the connection member 30 are assembled.

Engaging holes 27 (see FIG. 3) which engage with the device cover 24 are formed in the side wall surfaces 23b and 23c of the device case 23. In addition, a protrusion portion 28 which protrudes to the outside in the Y-axis direction is formed in the vicinity of the opening portion 25 on a pair of the side wall surfaces 23c facing the lateral direction (Y-axis direction). An inclined surface 28a which is inclined toward the opening portion 25 is formed on the protrusion portion 28. The inclined surface 28a reduces load (stress concentration) applied to the electrode sheet 10 from an angle at which the bottom surface 23a and the side wall surface 23c intersect.

As shown in FIG. 3, the connection member 30 connects the conductive portion 11 of the electrode sheet 10 to the contact portion 21 by holding the electrode sheet 10 between the device 20 and the connection member 30. The connection member 30 includes a facing portion 31 which faces the contact portion 21 with the electrode sheet 10 held therebetween, a fitting portion 32 which fits into the device 20, and an extending portion 33 which extends to a side of the electrode sheet 10 in the lateral direction (Y-axis direction) from the side edge surface of the electrode sheet 10.

The facing portion 31, the fitting portion 32, and the extending portion 33 are integrated by resin molding or the like. The connection member 30 is preferably made of a material having spring properties, and may be made of a metal material as long as insulating properties with the electrode sheet 10 can be ensured. As shown in FIG. 2, the facing portion 31 is formed in a rectangular flat plate shape extending along the Y-axis direction. A dimension of the facing portion 31 in the X-axis direction is set to a size which allows the facing portion 31 to be inserted into the opening portion 25 of the device case 23. At least a part of the facing portion 31 may be transparent. According to the configuration, connection status between the electrode sheet 10 and the contact portion 21 can be visualized.

The fitting portion 32 is formed in a pair at end parts of the facing portion 31 on both sides in the Y-axis direction. As shown in FIG. 3, the fitting portion 32 is formed in a substantially upside-down U shape which protrudes upward (+Z side). A fitting claw 32a is formed on a side surface of the fitting portion 32, facing the outside in the Y-axis direction. The fitting claw 32a has a shape of a corner portion formed by hypotenuse and base of a right triangle. The shape of the fitting claw 32a is an example, and a lower surface (bottom side) of the fitting claw 32a may be changed to an inclined surface in order to easily detach the connection member 30 from the device 20.

The fitting portion 32 is elastically deformed to the inside in the Y-axis direction, so that the fitting claw 32a can be moved to the inside in the Y-axis direction. The extending portion 33 is formed in a pair at end parts of the pair of the fitting portions 32 on the outside in the Y-axis direction. The extending portion 33 is formed in a flat plate shape extending along the Y-axis direction. The extending portion 33 extends to the side of the electrode sheet 10 in the lateral direction (Y-axis direction) from the side edge surface of the electrode sheet 10 in a state in which the fitting portion 32 fits into the device 20.

As shown in FIG. 3, the device 20 has a fitting target portion 29 into which the fitting portion 32 fits. The fitting target portion 29 is formed inside the opening portion 25 of the device case 23. Specifically, the fitting target portion 29 is formed on an inner surface side of a portion of the side wall surface 23c, where the protrusion portion 28 is formed. The fitting target portion 29 has a stepped shape which is recessed toward the outside in the Y-axis direction. The fitting target portion 29 has a flat surface portion which abuts the fitting claw 32a in the Z-axis direction.

In order to assemble the bioelectric potential measurement device 1 having the above-described configuration, first, as shown in FIG. 2, the pair of the fitting portions 32 of the connection member 30 are inserted into the pair of the through-holes 13 of the electrode sheet 10. As a result, the electrode sheet 10 can be connected to the device 20 in a state in which the electrode sheet 10 is positioned. Next, the pair of the fitting portions 32, which penetrate the electrode sheet 10, are inserted into the opening portion 25 formed in the bottom surface 23a of the device case 23.

In a case of passing through the opening portion 25, the pair of the fitting portions 32 are elastically deformed inward in the Y-axis direction by an oblique side portion as shown in FIG. 3. After passing through the opening portion 25, the pair of the fitting portions 32 are restored to their original shape, and fit into the fitting target portions 29 formed inside the opening portion 25. As a result, the connection member 30 is connected to the device 20 in a state in which the electrode sheet 10 is held therebetween.

The facing portion 31 of the connection member 30 connects the end parts of the first wiring portion 12A to the third wiring portion 12C of the conductive portion 11 to the first contact portion 21A to the third contact portion 21C of the device 20. Here, since the through-hole 13 of the electrode sheet 10 is provided in a pair and the first contact portion 21A to the third contact portion 21C are disposed between the pair of the through-holes 13 in a plan view, the misregistration between the electrode sheet 10 and the contact portion 21 can be suppressed. That is, by designing the positional relationship between the through-hole 13 and the fitting portion 32 with high accuracy, the misregistration can be suppressed. The gap between the through-hole 13 and the fitting portion 32 may be narrower than a misregistration amount tolerance between the end parts of the first wiring portion 12A to the third wiring portion 12C of the conductive portion 11 and the first contact portion 21A to the third contact portion 21C of the device 20. The misregistration amount tolerance refers to a size of a gap such that electrical conduction (connection) between the conductive portion 11 and the contact portion 21 can be ensured.

As described above, with the bioelectric potential measurement device 1 according to the present embodiment, since the electrode sheet 10 is held between the device 20 and the connection member 30, the electrode sheet 10 can be reliably connected to the contact portion 21 of the device 20. In addition, since the electrode sheet 10 has a shape (through-hole 13) corresponding to the fitting portion 32 of the connection member 30, it is not necessary to increase the sizes of the device 20 and the connection member 30 in accordance with the size of the electrode sheet 10.

As described above, the bioelectric potential measurement device 1 according to the present embodiment includes the electrode sheet 10 configured to acquire a biological signal, the device 20 which has the contact portion 21 connected to the electrode sheet 10 and the connection member 30 configured to connect the electrode sheet 10 to the contact portion 21 by holding the electrode sheet 10 between the device 20 and the connection member 30, in which the connection member 30 has the fitting portion 32 which fits into the device 20, and the electrode sheet 10 has a shape corresponding to the fitting portion 32. According to the configuration, the bioelectric potential measurement device 1 in which the electrode sheet 10 can be reliably connected to the device 20 without being affected by the size of the electrode sheet 10 is obtained.

In addition, in the bioelectric potential measurement device 1 according to the present embodiment, the device 20 has the fitting target portion 29 into which the fitting portion 32 fits. According to the configuration, the connection member 30 is less likely to come off the device 20.

In addition, in the bioelectric potential measurement device 1 according to the present embodiment, the pair of the fitting portions 32 are provided in at least the lateral direction (Y-axis direction) of the electrode sheet 10. According to the configuration, the size of the device 20 can be reduced in the longitudinal direction of the electrode sheet 10, as compared with a case where the pair of the fitting portions 32 are provided in the longitudinal direction (X-axis direction) of the electrode sheet 10.

In addition, in the bioelectric potential measurement device 1 according to the present embodiment, the plurality of the contact portions 21 are provided, and the connection member 30 connects the electrode sheet 10 to the plurality of the contact portions 21. According to the configuration, the electrode sheet 10 can be simultaneously connected to the plurality of the contact portions 21.

In addition, in the bioelectric potential measurement device 1 according to the present embodiment, a positioning mechanism which positions the electrode sheet 10 and the contact portion 21 is provided, and the positioning mechanism includes the fitting portion 32. According to the configuration, since the fitting portion 32 also serves as the positioning mechanism, the number of components of the bioelectric potential measurement device 1 can be reduced.

In addition, in the bioelectric potential measurement device 1 according to the present embodiment, the positioning mechanism includes a through-hole 13 which is formed in the electrode sheet 10 corresponding to the fitting portion 32 and through which the fitting portion 32 is disposed. According to the configuration, since the fitting portion 32 of the connection member 30 is inserted through the through-hole 13 formed in the electrode sheet 10 and fits into the device 20, an outer shape of the electrode sheet 10 can be freely expanded.

In addition, in the bioelectric potential measurement device 1 according to the present embodiment, the through-hole 13 is provided in a pair, and the contact portion 21 is disposed between the pair of the through-holes 13 in a plan view. According to the configuration, since the pair of the fitting portions 32 of the connection member 30 is inserted into the pair of the through-holes 13 formed in the electrode sheet 10, the electrode sheet 10 can be positioned with high accuracy with respect to the contact portion 21.

In addition, in the bioelectric potential measurement device 1 according to the present embodiment, the connection member 30 includes the extending portion 33 which extends laterally from the side edge surface of the electrode sheet 10 in the lateral direction of the electrode sheet 10. According to the configuration, the bioelectric potential measurement device 1 (particularly, the electrode sheet 10 which is pressure-bonded to the skin) is easily detachable from the living body by hooking a finger on the extending portion 33.

In addition, in the bioelectric potential measurement device 1 according to the present embodiment, the electrode sheet 10 is a transparent electrode. According to the configuration, visibility is improved by providing the transparent electrode as the electrode sheet 10, and thus the misregistration is easily confirmed.

### Second Embodiment

Next, a second embodiment of the present invention will be described. In the following description, the same or equivalent configurations as those in the above-described embodiment are denoted by the same reference numerals, and descriptions thereof will be simplified or not be repeated.

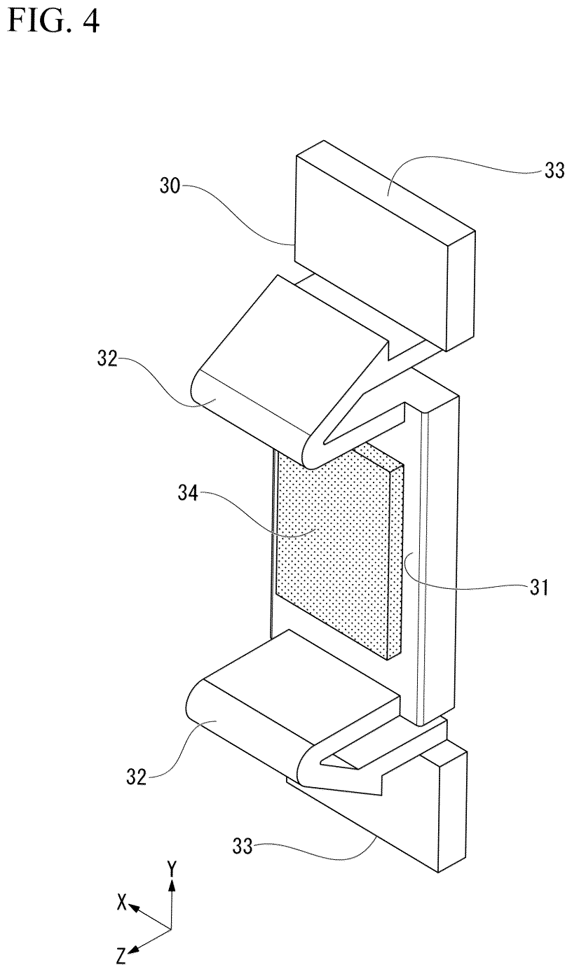

FIG. 4 is a perspective view of the connection member 30 according to the second embodiment.

As shown in FIG. 4, the connection member 30 according to the second embodiment includes an elastic portion 34 in the facing portion 31. The elastic portion 34 may be, for example, an elastic material softer than the facing portion 31, and is formed of, for example, rubber, an elastomer, or the like. The elastic portion 34 may be disposed in a range which covers at least the first contact portion 21A to the third contact portion 21C of the device 20 (that is, the end parts of the first wiring portion 12A to the third wiring portion 12C of the electrode sheet 10).

As described above, the connection member 30 according to the second embodiment includes the elastic portion 34 in the facing portion 31 which faces the contact portion 21 with the electrode sheet 10 held therebetween. According to the configuration, the electrode sheet 10 can be reliably connected to the contact portion 21 by pressing the electrode sheet 10 with the elastic portion 34.

### Third Embodiment

Next, a third embodiment of the present invention will be described. In the following description, the same or equivalent configurations as those in the above-described embodiment are denoted by the same reference numerals, and descriptions thereof will be simplified or not be repeated.

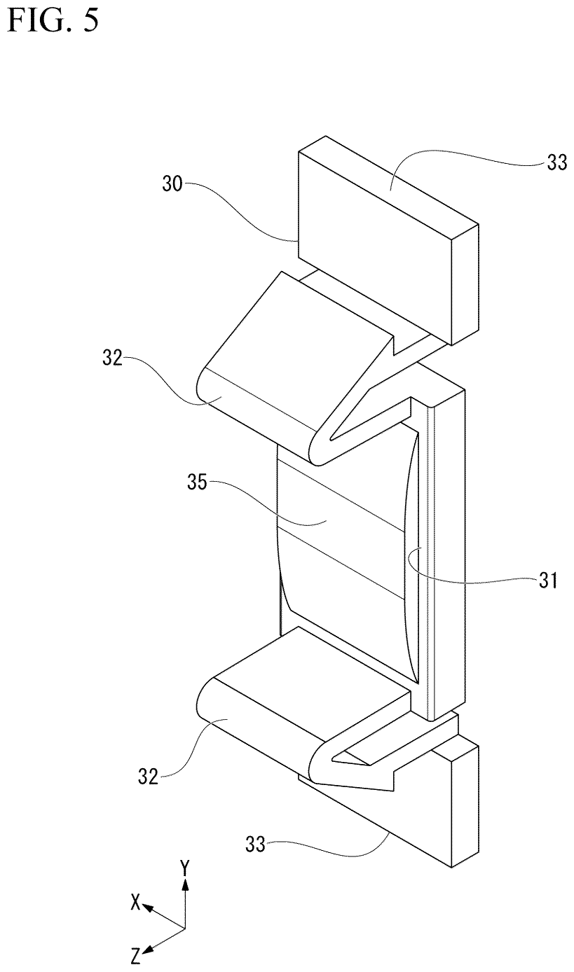

FIG. 5 is a perspective view of the connection member 30 according to the third embodiment.

As shown in FIG. 5, the connection member 30 according to the third embodiment includes a projected portion 35 in the facing portion 31. The projected portion 35 protrudes upward (+Z side) toward the electrode sheet 10. The projected portion 35 is formed in a semicircular or dome shape, in which a central portion in the Y-axis direction is a planar portion and which is gently inclined from the planar portion toward the outside in the Y-axis direction. The planar portion of the projected portion 35 may be disposed in a range which covers at least the first contact portion 21A to the third contact portion 21C of the device 20 (that is, the end parts of the first wiring portion 12A to the third wiring portion 12C of the electrode sheet 10).

As described above, the connection member 30 according to the third embodiment includes the projected portion 35 which protrudes toward the electrode sheet 10, in the facing portion 31 which faces the contact portion 21 with the electrode sheet 10 held therebetween. According to the configuration, the electrode sheet 10 can be reliably connected to the contact portion 21 by pressing the electrode sheet 10 with the projected portion 35.

### Fourth Embodiment

Next, a fourth embodiment of the present invention will be described. In the following description, the same or equivalent configurations as those in the above-described embodiment are denoted by the same reference numerals, and descriptions thereof will be simplified or not be repeated.

FIG. 6 is a perspective view of the connection member 30 according to the fourth embodiment.

As shown in FIG. 6, the connection member 30 according to the fourth embodiment includes a recessed portion 36 in the facing portion 31. The recessed portion 36 is recessed toward a side (−Z side) opposite to the electrode sheet 10. The recessed portion 36 is formed in a groove shape which is rectangular as viewed from the Z-axis direction. The recessed portion 36 may be disposed in a range which covers at least the first contact portion 21A to the third contact portion 21C of the device 20 (that is, the end parts of the first wiring portion 12A to the third wiring portion 12C of the electrode sheet 10).

As described above, the connection member 30 according to the fourth embodiment includes the recessed portion 36 which is recessed toward a side opposite to the electrode sheet 10, in the facing portion 31 which faces the contact portion 21 with the electrode sheet 10 held therebetween. According to the configuration, the recessed portion 36 can suppress the pressing of the electrode sheet 10 against the contact portion 21 protruding from the substrate 22 with an excessive force.

### Fifth Embodiment

Next, a fifth embodiment of the present invention will be described. In the following description, the same or equivalent configurations as those in the above-described embodiment are denoted by the same reference numerals, and descriptions thereof will be simplified or not be repeated.

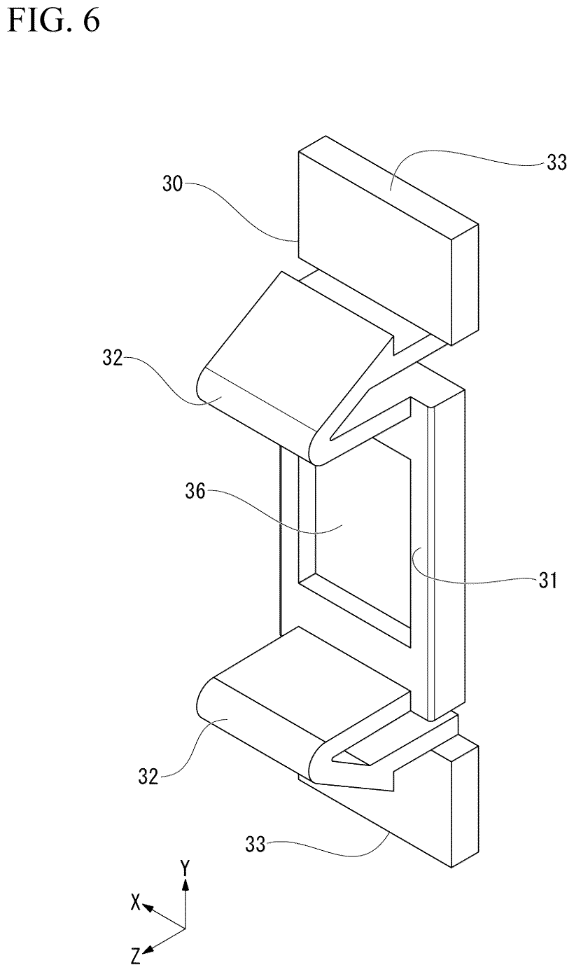

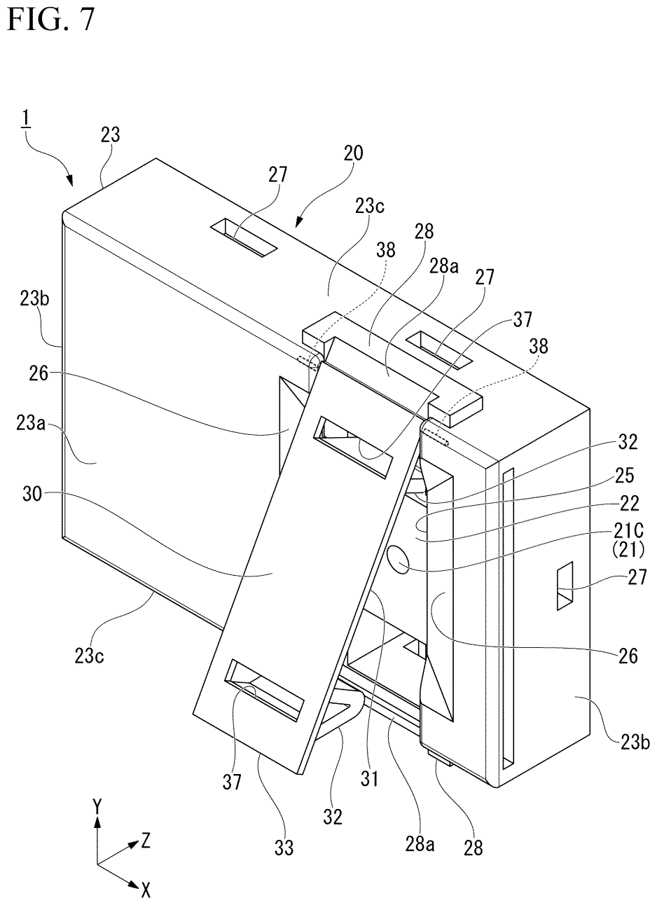

FIG. 7 is a perspective view of the bioelectric potential measurement device 1 according to the fifth embodiment. In FIG. 7, the electrode sheet 10 is not shown in order to improve visibility of the connection member 30.

As shown in FIG. 7, in the bioelectric potential measurement device 1 according to the fifth embodiment, the connection member 30 which is movable is integrated with the device 20.

The connection member 30 shown in FIG. 7 includes a rotating shaft 38 which is pivotally supported by the device case 23. The rotating shaft 38 protrudes from an end part of the connection member 30 on the +Y side to both sides in the X-axis direction. In the device case 23, a bearing hole into which the rotating shaft 38 is inserted is formed in the vicinity of an end part of the opening portion 25 on the +Y side. Accordingly, the connection member 30 is integrated with the device 20 to be rotatable around an axis extending in the X-axis direction.

The connection member 30 includes the facing portion 31, the fitting portion 32, and the extending portion 33, in addition to the rotating shaft 38. The extending portion 33 is formed on a side (only one side) of the connection member 30 opposite to the rotating shaft 38. The extending portion 33 and the facing portion 31 are connected to each other at two positions on both sides of a slit 37 formed in the connection member 30. The slit 37 is formed at a position corresponding to the fitting portion 32.

As described above, in the bioelectric potential measurement device 1 according to the fifth embodiment, the connection member 30 which is movable is integrated with the device 20. According to the configuration, since the device 20 and the connection member 30 are integrated, the connection member 30 can be prevented from being lost. In addition, since the device 20 and the connection member 30 are connected to each other on a predetermined trajectory, connection stability between the device 20 and the connection member 30 can be improved.

### Sixth Embodiment

Next, a sixth embodiment of the present invention will be described. In the following description, the same or equivalent configurations as those in the above-described embodiment are denoted by the same reference numerals, and descriptions thereof will be simplified or not be repeated.

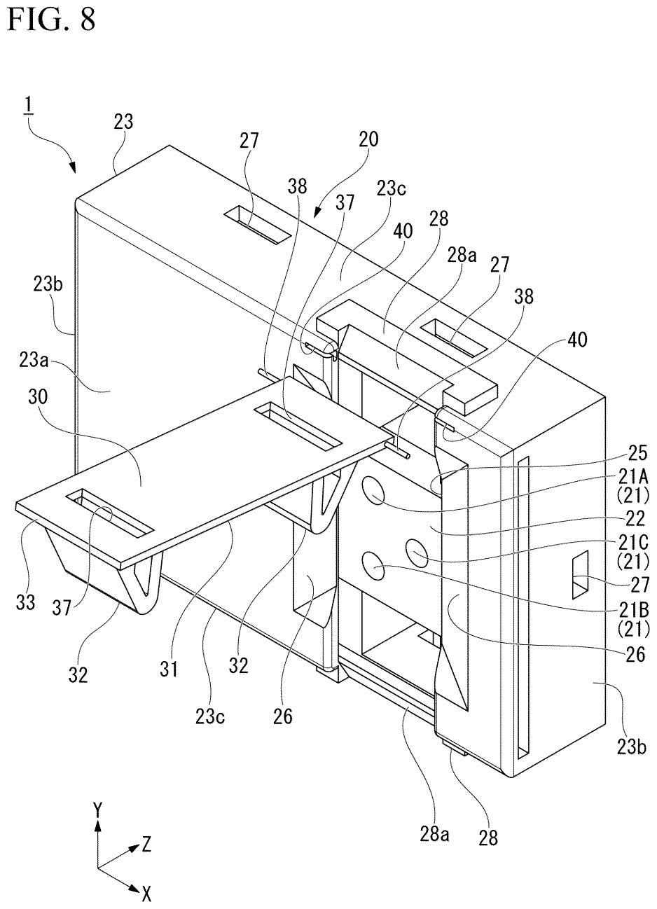

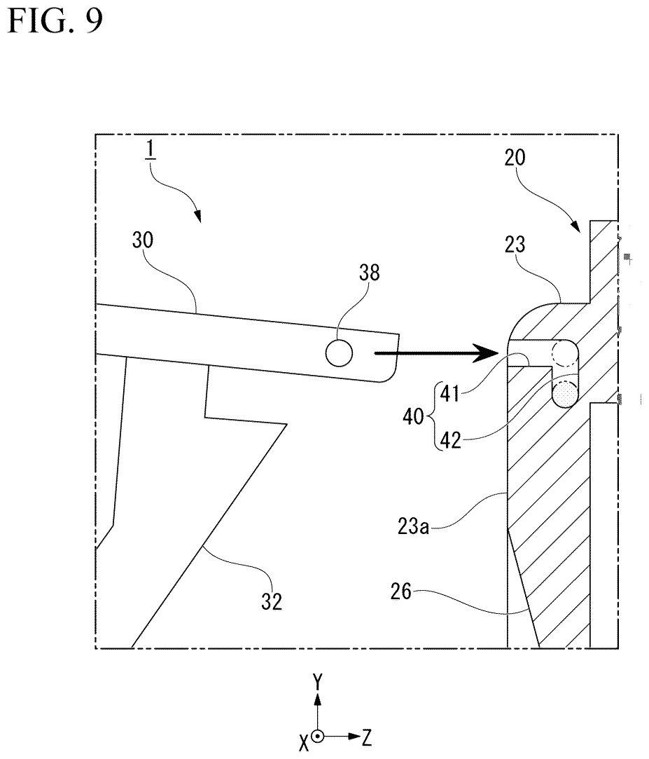

FIG. 8 is a perspective view of the bioelectric potential measurement device 1 according to the sixth embodiment. In FIG. 8, the electrode sheet 10 is not shown in order to improve visibility of the connection member 30. FIG. 9 is a cross-sectional view of a main part of the bioelectric potential measurement device 1 according to the sixth embodiment.

As shown in these drawings, in the bioelectric potential measurement device 1 according to the sixth embodiment, the connection member 30 is integrated with the device 20 to be separable.

In the device case 23, an insertion hole 40 into which the rotating shaft 38 is inserted is formed in the vicinity of an end part of the opening portion 25 on the +Y side. As shown in FIG. 9, the insertion hole 40 includes an insertion portion 41 and an engaging portion 42. The insertion portion 41 linearly extends in the Z-axis direction from the bottom surface 23a of the device case 23. The engaging portion 42 is bent at a right angle with respect to the insertion portion 41, and linearly extends in the Y-axis direction.

The rotating shaft 38 moves in the Z-axis direction and the Y-axis direction, and engages with the insertion hole 40 having an L shape in cross section. As a result, the connection member 30 is integrated with the device 20 to be rotatable around an axis extending in the X-axis direction. In a case where the connection member 30 is detached from the device 20, the connection member 30 is moved in the Y-axis direction and the Z-axis direction, and the rotating shaft 38 is pulled out from the insertion hole 40 having an L shape in cross section.

As described above, in the bioelectric potential measurement device 1 according to the sixth embodiment, the connection member 30 is integrated with the device 20 to be separable. According to the configuration, the connection member 30 can be detached from the device 20 except for a case of use. In addition, in a case of use, since the device 20 and the connection member 30 can be integrated, the connection stability between the device 20 and the connection member 30 can be improved.

### Seventh Embodiment

Next, a seventh embodiment of the present invention will be described. In the following description, the same or equivalent configurations as those in the above-described embodiment are denoted by the same reference numerals, and descriptions thereof will be simplified or not be repeated.

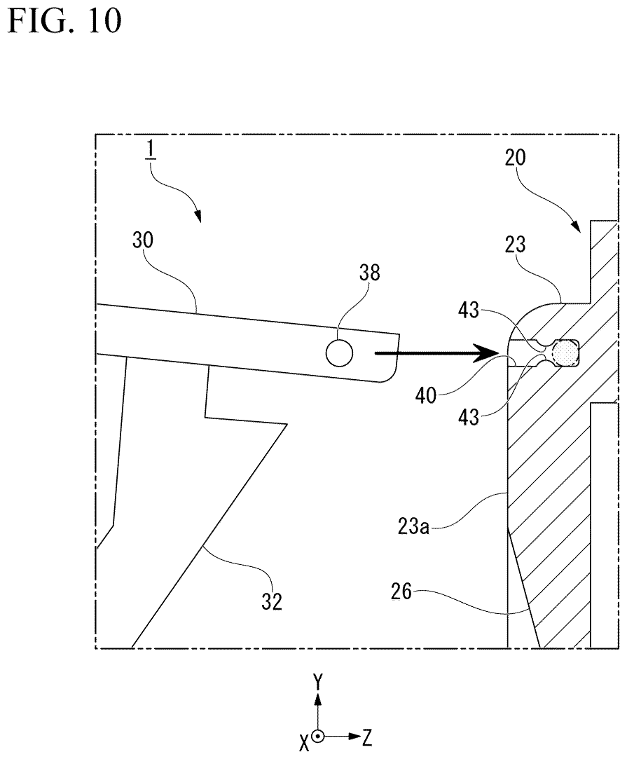

FIG. 10 is a cross-sectional view of a main part of the bioelectric potential measurement device 1 according to the seventh embodiment. FIG. 10 is a cross-sectional view of the same portion as that of FIG. 9 described above.

As shown in FIG. 10, a pair of protrusion portions 43 are formed in the insertion hole 40 according to the seventh embodiment.

The insertion hole 40 shown in FIG. 10 is not bent in an L-shape, and linearly extends in the Z-axis direction. The pair of the protrusion portions 43 are formed on an inner wall surface of the insertion hole 40 facing in the Y-axis direction. A gap between the pair of the protrusion portions 43 is slightly narrower than a diameter of the rotating shaft 38. Therefore, the rotating shaft 38 can be prevented from easily coming out of the insertion hole 40 after passing through the pair of the protrusion portions 43.

As described above, in the bioelectric potential measurement device 1 according to the seventh embodiment, the connection member 30 is integrated with the device 20 to be separable. According to the configuration, the connection member 30 can be detached from the device 20 except for a case of use. In addition, in a case of use, the device 20 and the connection member 30 are connected to each other on a predetermined trajectory, and thus the connection stability between the device 20 and the connection member 30 can be improved.

### Eighth Embodiment

Next, an eighth embodiment of the present invention will be described. In the following description, the same or equivalent configurations as those in the above-described embodiment are denoted by the same reference numerals, and descriptions thereof will be simplified or not be repeated.

FIGS. 11A and 11B are simplified views of showing the bioelectric potential measurement device 1 according to the eighth embodiment. FIG. 11A shows a state before the conductive portion 11 of the electrode sheet 10 is connected to the contact portion 21 of the device 20. FIG. 11B shows a state after the conductive portion 11 of the electrode sheet 10 is connected to the contact portion 21 of the device 20.

As shown in FIGS. 11A and 11B, the connection member 30 according to the eighth embodiment is provided with an abutting portion 50 which abuts the device 20 through a portion (sheet substrate portion other than the conductive portion 11) of the electrode sheet 10, which does not overlap with the contact portion 21.

The abutting portion 50 is formed in a convex shape protruding from the facing portion 31 of the connection member 30 toward the device 20 side. A distal end surface of the abutting portion 50 is flat. As shown in FIG. 11B, the abutting portion 50 abuts the device 20 through the sheet substrate portion of the electrode sheet 10 in a state of being held between the device 20 and the connection member 30.

As described above, the connection member 30 (one) according to the eighth embodiment is provided with the abutting portion 50 which abuts the device 20 (the other) through the portion of the electrode sheet 10, which does not overlap with the contact portion 21. According to the configuration, the abutting portion 50 can serve as a spacer so that an excessive force is not applied to the contact portion 21.

The abutting portion 50 may be provided on both sides of the connection member 30 and the device 20; and in a case where the contact portion 21 does not protrude downward and the conductive portion 11 protrudes upward from the electrode sheet 10, the abutting portion 50 may be provided only on the device 20 side.

### Ninth Embodiment

Next, a ninth embodiment of the present invention will be described. In the following description, the same or equivalent configurations as those in the above-described embodiment are denoted by the same reference numerals, and descriptions thereof will be simplified or not be repeated.

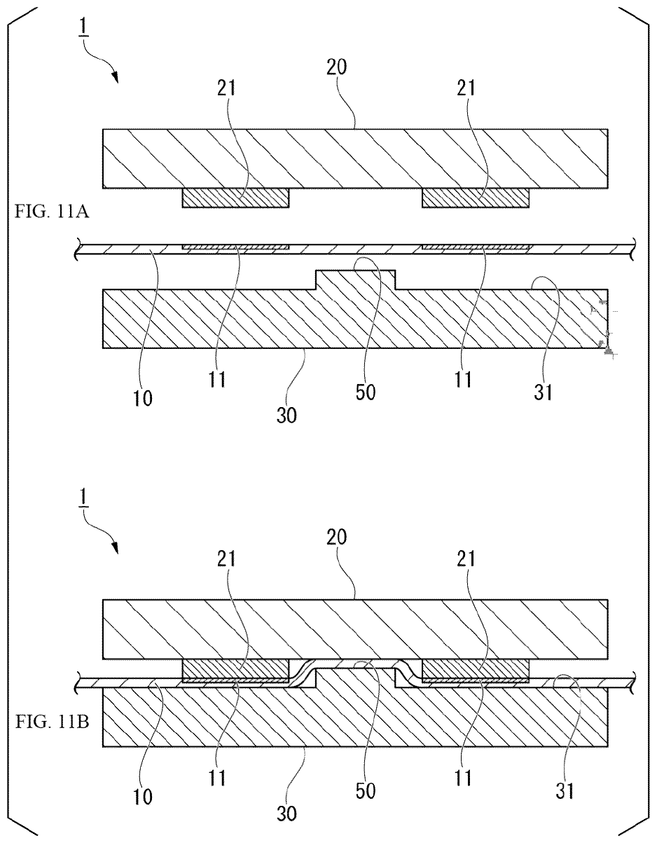

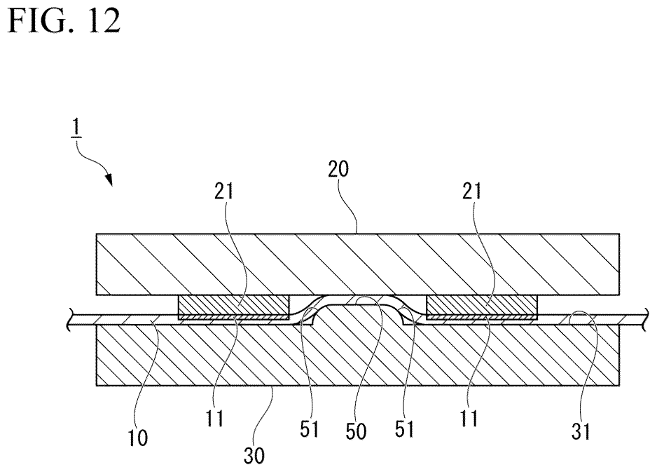

FIG. 12 is a simplified view of the bioelectric potential measurement device 1 according to the ninth embodiment.

As shown in FIG. 12, the abutting portion 50 according to the ninth embodiment has a curved corner portion 51. Specifically, in the abutting portion 50, a corner portion 51 where a distal end surface and a side wall surface intersect with each other is formed in an arc shape in a longitudinal cross-sectional view along an up-down direction of the abutting portion 50.

As described above, the abutting portion 50 according to the ninth embodiment has the curved corner portion 51. According to the configuration, it is possible to reduce load (stress concentration) applied to the electrode sheet 10 from the corner portion 51 of the abutting portion 50.

### Tenth Embodiment

Next, a tenth embodiment of the present invention will be described. In the following description, the same or equivalent configurations as those in the above-described embodiment are denoted by the same reference numerals, and descriptions thereof will be simplified or not be repeated.

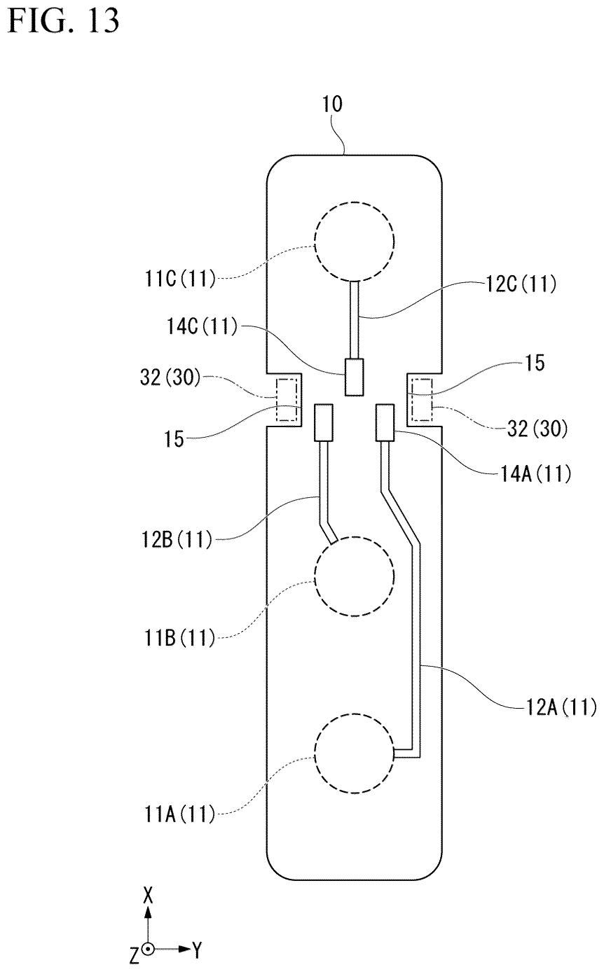

FIG. 13 is a plan view of the electrode sheet 10 according to the tenth embodiment.

As shown in FIG. 13, the electrode sheet 10 according to the tenth embodiment has a constricted portion 15. Specifically, the constricted portion 15 is formed in a pair on an outer edge of the electrode sheet 10 in the lateral direction (Y-axis direction).

The electrode sheet 10 has a plurality of the conductive portions 11. The conductive portion 11 includes terminal portions (first terminal portion 14A to third terminal portion 14C) which are in contact with the contact portion 21 (not shown in FIG. 13) of the device 20, electrode portions (first electrode portion 11A to third electrode portion 11C) which are in contact with the biological side, and wiring portion (first wiring portion 12A to third wiring portion 12C) which connect the terminal portion and the electrode portion to each other. The first terminal portion 14A to the third terminal portion 14C are disposed between a pair of the constricted portions 15.

The fitting portion 32 of the connection member 30 is disposed to be inserted into the constricted portion 15 in the Z-axis direction. The constricted portion 15 constitutes a positioning mechanism which positions the electrode sheet 10 with respect to the device 20, together with the fitting portion 32. According to the configuration, even in a case where a width of the electrode sheet 10 is narrow and the above-described through-hole 13 (see FIG. 2) cannot be formed, the electrode sheet 10 can be positioned with respect to the contact portion 21 by the constricted portion 15.

In addition, the first terminal portion 14A to the third terminal portion 14C are formed to be long in a second direction (X-axis direction) intersecting with the first direction (Y-axis direction). According to the configuration, the first terminal portion 14A to the third terminal portion 14C are extended in the second direction (X-axis direction) in which the stretch and contraction of the electrode sheet 10 is not restricted by the pair of the fitting portions 32, whereby the connection between the first terminal portion 14A to the third terminal portion 14C and the first contact portion 21A to the third contact portion 21C can be stabilized. In addition, the second direction intersecting with the first direction is not limited to a direction intersecting with the first direction at a right angle, and includes, for example, a direction intersecting with the first direction at 60° or more and 120° or less.

### Eleventh Embodiment

Next, an eleventh embodiment of the present invention will be described. In the following description, the same or equivalent configurations as those in the above-described embodiment are denoted by the same reference numerals, and descriptions thereof will be simplified or not be repeated.

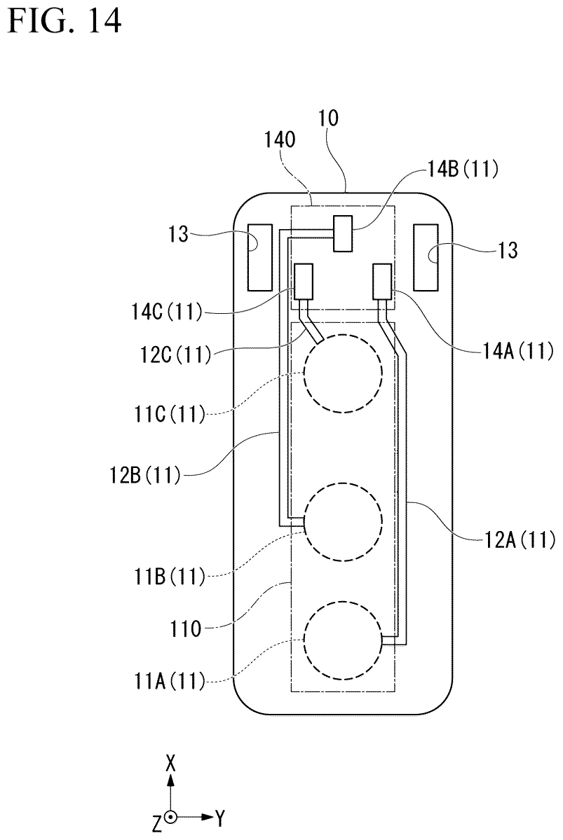

FIG. 14 is a plan view of the electrode sheet 10 according to the eleventh embodiment.

As shown in FIG. 14, in the electrode sheet 10 according to the eleventh embodiment, a terminal group 140 including a plurality of the conductive portions 11 and a plurality of terminal portions (first terminal portion 14A to third terminal portion 14C) and an electrode group 110 including a plurality of electrode portions (first electrode portion 11A to third electrode portion 11C) are arranged to be spaced from each other in a plan view. That is, the eleventh embodiment is not configured such that the first terminal portion 14A to the third terminal portion 14C are disposed between the second electrode portion 11B and the third electrode portion 11C, as shown in FIG. 13.

According to the configuration, since the electrode group 110 to be connected to the biological side is disposed to be separated from the terminal group 140 to be connected to the device 20 side by the connection member 30, it is possible to suppress peeling of the electrode group 110 from the biological side. That is, the vicinity of the terminal group 140 is affected by floating of the electrode sheet 10 from the biological side, caused by the thickness of the connection member 30 in the Z-axis direction, and thus the electrode sheet 10 is likely to be peeled off. Therefore, by separating the electrode group 110 from the terminal group 140, it is possible to suppress the peeling of the electrode group 110 from the biological side.

In addition, in the electrode group 110, the plurality of the electrode portions (the first electrode portion 11A to the third electrode portion 11C) are linearly arranged in the X-axis direction, and the terminal group 140 is disposed on an extension line of the plurality of the electrode portions (the first electrode portion 11A to the third electrode portion 11C) in the X-axis direction. In the configuration, the electrode sheet 10 can be reduced in size.

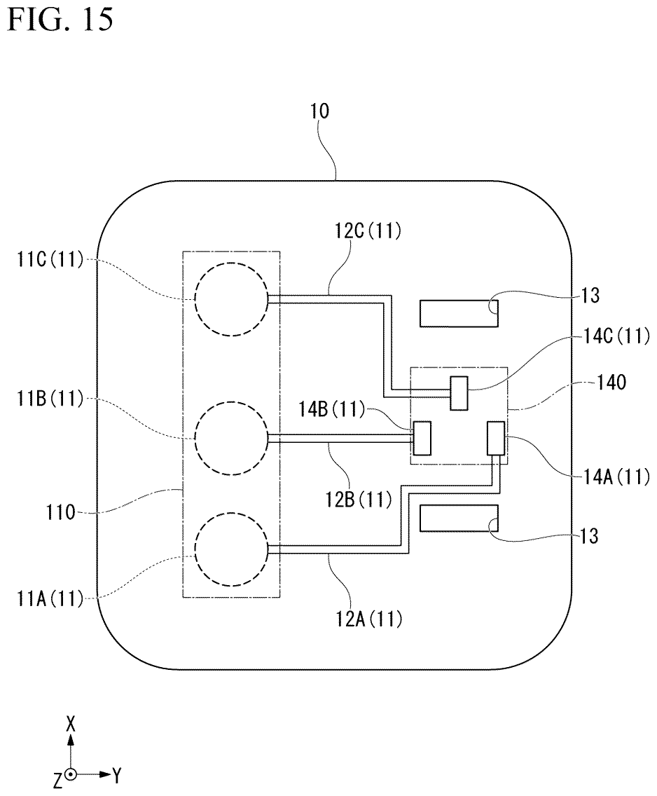

FIG. 15 is a plan view of the electrode sheet 10 according to a comparative example of the eleventh embodiment.

In the electrode sheet 10 shown in FIG. 15, the terminal group 140 is not disposed on the extension line of the plurality of the electrode portions (the first electrode portion 11A to the third electrode portion 11C). In this case, the size of the electrode sheet 10 is larger than that of the electrode sheet 10 shown in FIG. 14. However, there is an advantage that the peeling from the biological side due to the thickness of the connection member 30 in the Z-axis direction can be reduced by the separation of the electrode group 110 and the terminal group 140.

### Twelfth Embodiment

Next, a twelfth embodiment of the present invention will be described. In the following description, the same or equivalent configurations as those in the above-described embodiment are denoted by the same reference numerals, and descriptions thereof will be simplified or not be repeated.

FIG. 16 is a perspective view of the connection member 30 according to the twelfth embodiment. As shown in FIG. 16, the connection member 30 according to the twelfth embodiment includes a first fitting portion 32A which is provided in a pair in the lateral direction (Y-axis direction) of the electrode sheet 10, and a second fitting portion 32B which fits into the device 20 at a position different from the first fitting portion 32A.

The second fitting portion 32B shown in FIG. 16 is provided in parallel with the first fitting portion 32A. According to the configuration, since the number of fitting points with the device 20 is increased, fitting stability of the connection member 30 can be increased. That is, the connection member 30 is even less likely to come off the device 20.

### Thirteenth Embodiment

Next, a thirteenth embodiment of the present invention will be described. In the following description, the same or equivalent configurations as those in the above-described embodiment are denoted by the same reference numerals, and descriptions thereof will be simplified or not be repeated.

FIG. 17 is a perspective view of the connection member 30 according to the thirteenth embodiment.

As shown in FIG. 17, the second fitting portion 32B according to the thirteenth embodiment is provided in an orientation different from the first fitting portion 32A.

Specifically, the second fitting portion 32B is provided in a direction (X-axis direction) in which the longitudinal direction of the electrode sheet 10 extends. As the second fitting portion 32B fits into the device 20, in a case where a force is applied in a direction around an axis extending in the lateral direction (Y-axis direction) of the electrode sheet 10 with the first fitting portion 32A as the axis, for example, in a case where a finger is caught at an end part of the device 20 in the longitudinal direction (X-axis direction) during intense exercise, the rotation of the device 20 can be restricted by the fitting of the second fitting portion 32B.

### Fourteenth Embodiment

Next, a fourteenth embodiment of the present invention will be described. In the following description, the same or equivalent configurations as those in the above-described embodiment are denoted by the same reference numerals, and descriptions thereof will be simplified or not be repeated.

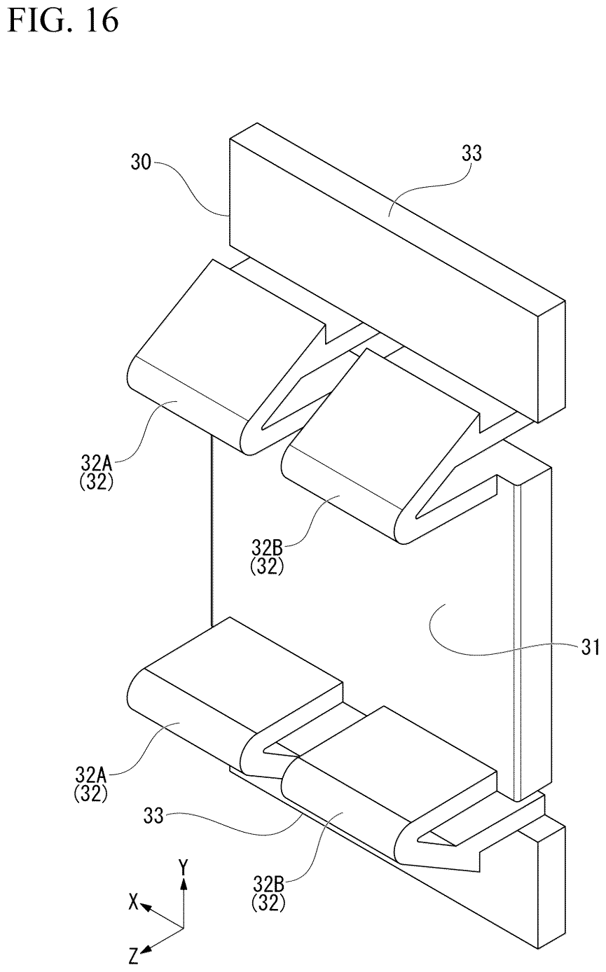

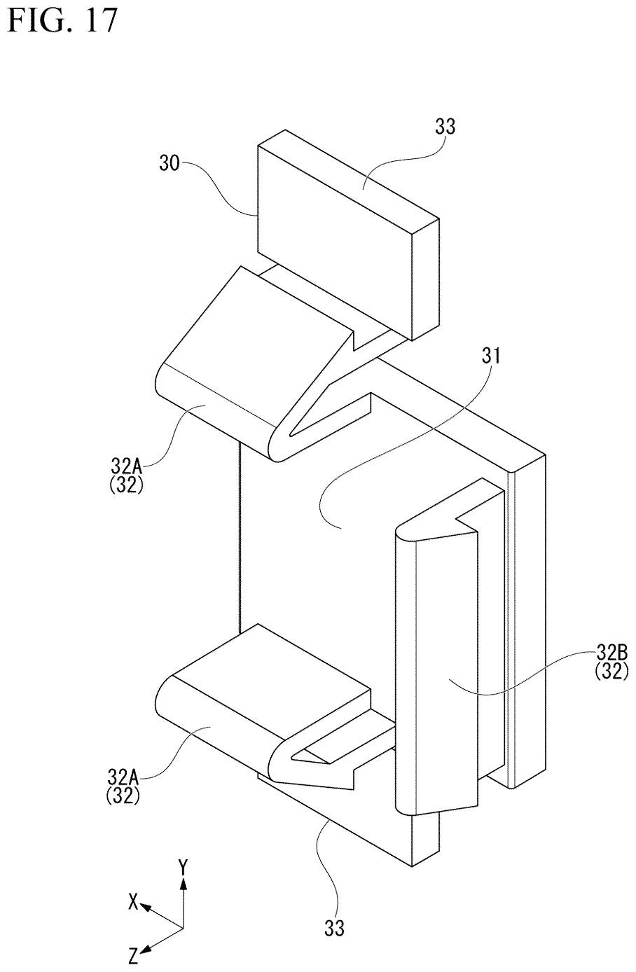

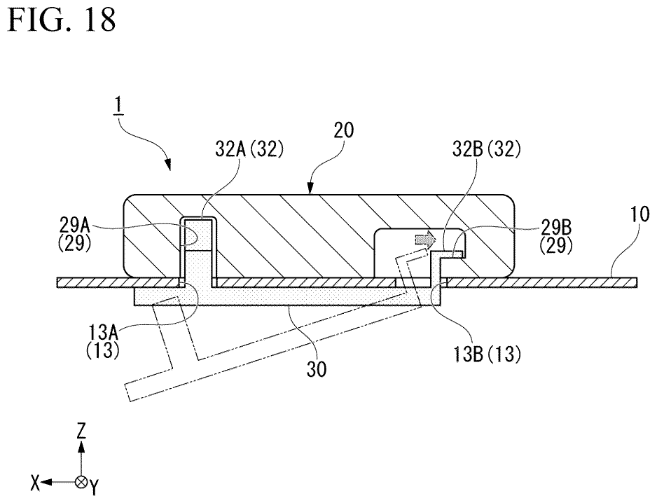

FIG. 18 is a schematic cross-sectional view of the bioelectric potential measurement device 1 according to the fourteenth embodiment along the longitudinal direction.

As shown in FIG. 18, the second fitting portion 32B according to the fourteenth embodiment is configured to slide in the longitudinal direction (X-axis direction) of the electrode sheet 10 as indicated by an arrow in the drawing, and to fit into the device 20.

Specifically, the device 20 includes a first fitting target portion 29A (fitted portion 29 shown in FIG. 3) into which the first fitting portion 32A fits, and a second fitting target portion 29B into which the second fitting portion 32B fits. The second fitting target portion 29B is formed in an upside-down L shape. The electrode sheet 10 includes a first through-hole 13A (same as the through-hole 13 shown in FIG. 3) into which the first fitting portion 32A is inserted, and a second through-hole 13B into which the second fitting portion 32B is inserted.

According to the configuration, the second fitting portion 32B can be fitted to the second fitting target portion 29B by inserting the second fitting portion 32B into the second fitting target portion 29B and sliding the second fitting portion 32B in the longitudinal direction (X-axis direction) of the electrode sheet 10. Thereafter, the first fitting portion 32A can be fitted to the first fitting target portion 29A by rotating the connection member 30 with the second fitting portion 32B fitted to the second fitting target portion 29B as a fulcrum.

In the configurations shown in FIGS. 16 and 17 described above, the first fitting portion 32A and the second fitting portion 32B need to be bent at the same time to be fitted to the device 20. However, in the configuration shown in FIG. 18, the first fitting portion 32A and the second fitting portion 32B can be individually fitted to the device 20, so that the connection member 30 can be easily fitted to the device 20.

### Fifteenth Embodiment

Next, a fifteenth embodiment of the present invention will be described. In the following description, the same or equivalent configurations as those in the above-described embodiment are denoted by the same reference numerals, and descriptions thereof will be simplified or not be repeated.

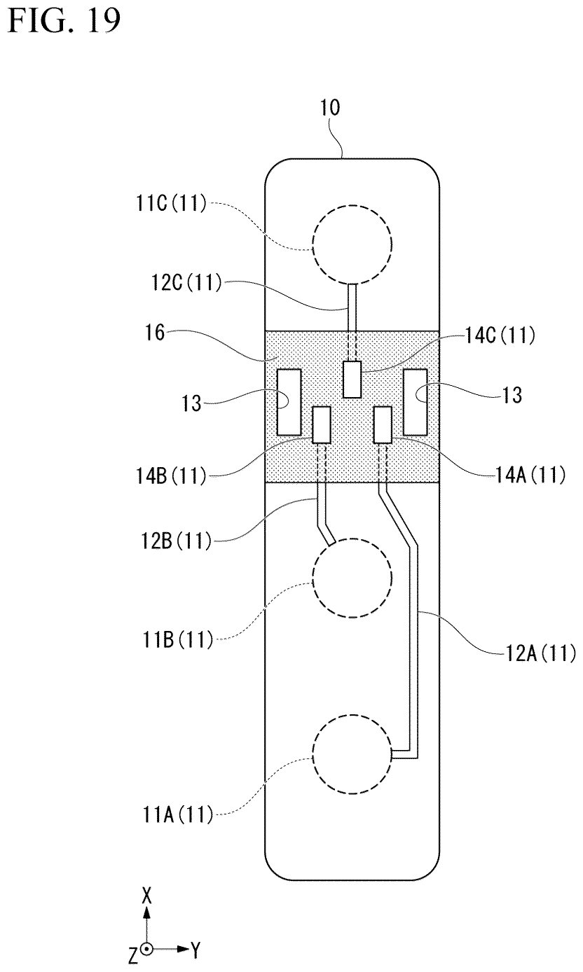

FIG. 19 is a plan view of the electrode sheet 10 according to the fifteenth embodiment.

As shown in FIG. 19, the electrode sheet 10 according to the fifteenth embodiment includes a shape holding portion 16 which is harder than a base material of the electrode sheet 10.

For example, in a case where the base material of the electrode sheet 10 is formed of a soft urethane sheet, the shape holding portion 16 is formed of a polyimide film or a PET film, which is harder than the urethane sheet. The shape holding portion 16 is disposed in the vicinity of the contact portion 21 of the device 20, and an opening through which the terminal portions (the first terminal portion 14A to the third terminal portion 14C) and the through-hole 13 are exposed is formed. According to the configuration, since twisting or deformation of the electrode sheet 10 in the vicinity of the contact portion 21 of the device 20 can be suppressed, stabilization of the positioning of the electrode sheet 10 and the contact portion 21 can be achieved. The shape holding portion 16 may be hardened by being thicker than the base material of the electrode sheet 10.

### Sixteenth Embodiment

Next, a sixteenth embodiment of the present invention will be described. In the following description, the same or equivalent configurations as those in the above-described embodiment are denoted by the same reference numerals, and descriptions thereof will be simplified or not be repeated.

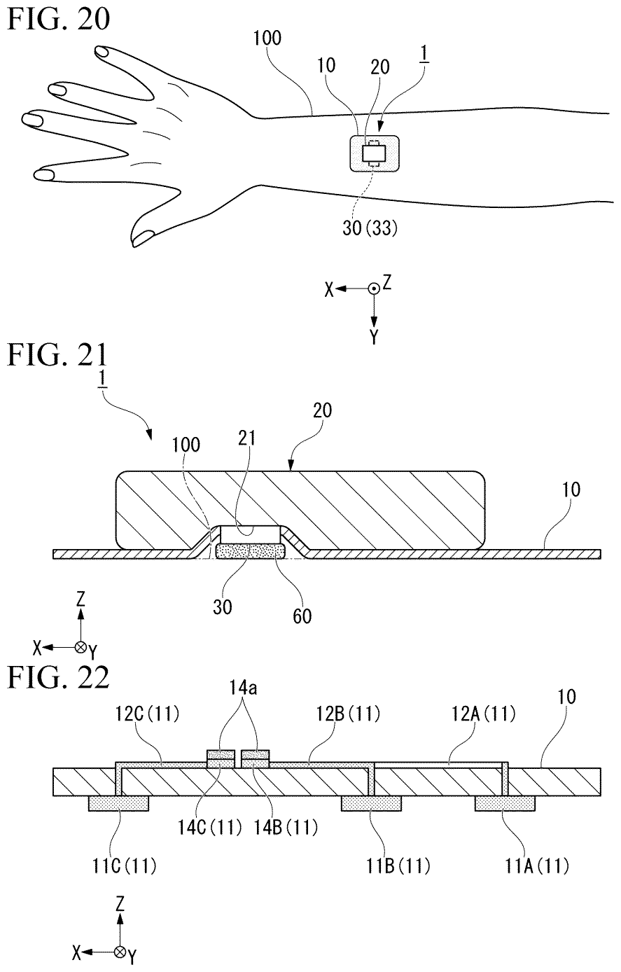

FIG. 20 is a view representing an example of use of a bioelectric potential measurement device 1 according to the sixteenth embodiment.

As shown in FIG. 20, in the bioelectric potential measurement device 1 according to the sixteenth embodiment, the connection member 30 is disposed inside an outer edge of the electrode sheet 10 in a plan view.

That is, in the sixteenth embodiment, the entire connection member 30 is covered with the electrode sheet 10, and the extending portion 33 does not protrude from the electrode sheet 10, which is different from the first embodiment shown in FIG. 1. According to the configuration, even in a case where the subject performs intense exercise, the finger or the like is less likely to be caught in the connection member 30 (extending portion 33), and thus the probability that the bioelectric potential measurement device 1 is unintentionally peeled off from the living body 100 is reduced.

### Seventeenth Embodiment

Next, a seventeenth embodiment of the present invention will be described. In the following description, the same or equivalent configurations as those in the above-described embodiment are denoted by the same reference numerals, and descriptions thereof will be simplified or not be repeated.

FIG. 21 is a schematic cross-sectional view of the bioelectric potential measurement device 1 according to the seventeenth embodiment along the longitudinal direction.

As shown in FIG. 21, the bioelectric potential measurement device 1 according to the seventeenth embodiment includes an adhesive portion 60 on the biological side of the connection member 30.

The adhesive portion 60 is applied to a surface of the connection member 30 facing a side opposite to the contact portion 21. A material of the adhesive portion 60 is not particularly limited as long as the adhesive portion 60 can adhere the connection member 30 to the biological side. According to the configuration, since not only the electrode sheet 10 but also the connection member 30 can be pressure-bonded to the biological side, the peeling of the electrode sheet 10 from the biological side with the connection member 30 as a starting point can be suppressed.

### Eighteenth Embodiment

Next, an eighteenth embodiment of the present invention will be described. In the following description, the same or equivalent configurations as those in the above-described embodiment are denoted by the same reference numerals, and descriptions thereof will be simplified or not be repeated.

FIG. 22 is a schematic cross-sectional view taken along a longitudinal direction of the electrode sheet 10 according to the eighteenth embodiment.

As shown in FIG. 22, in the electrode sheet 10 according to the eighteenth embodiment, a cured layer 14a is formed on a surface of the terminal portion (the first terminal portion 14A (not shown), the second terminal portion 14B, and the third terminal portion 14C).

The cured layer 14a is harder than the wiring portion (the first wiring portion 12A to the third wiring portion 12C). The cured layer 14a may be formed by modifying the surface of the terminal portion (the first terminal portion 14A to the third terminal portion 14C), or may be formed by coating the surface with an organic conductive material such as carbon, metal vapor deposition, or the like. According to the configuration, since the surface of the terminal portion is hardened, the terminal portion can be reliably connected to the contact portion 21, so that the connection between the terminal portion and the contact portion 21 can be stabilized.

### Nineteenth Embodiment

Next, a nineteenth embodiment of the present invention will be described. In the following description, the same or equivalent configurations as those in the above-described embodiment are denoted by the same reference numerals, and descriptions thereof will be simplified or not be repeated.

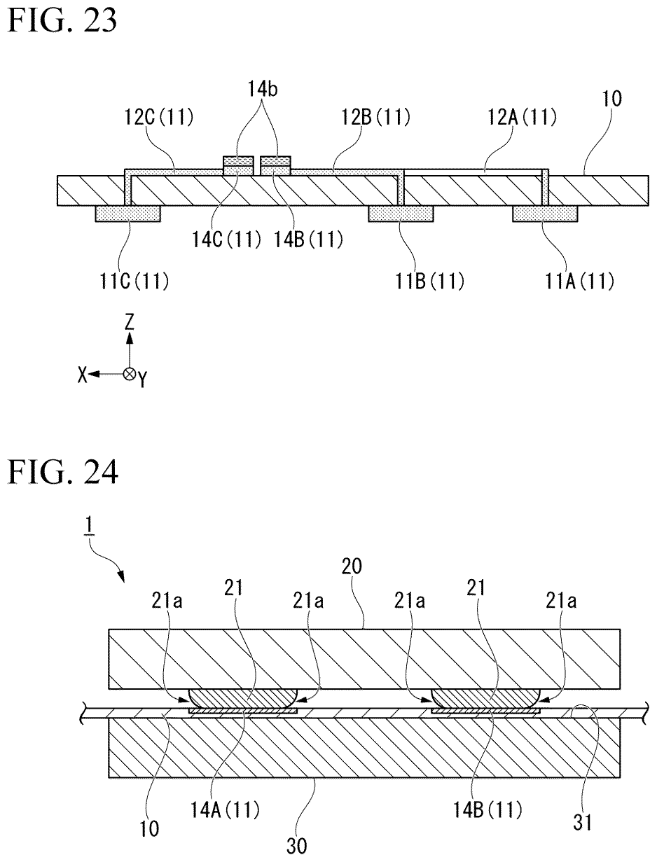

FIG. 23 is a schematic cross-sectional view taken along a longitudinal direction of the electrode sheet 10 according to the nineteenth embodiment. As shown in FIG. 23, in the electrode sheet 10 according to the nineteenth embodiment, a softening layer 14b is formed on a surface of the terminal portion (the first terminal portion 14A (not shown), the second terminal portion 14B, and the third terminal portion 14C).

The softening layer 14b has a hardness equal to or lower than the wiring portion (the first wiring portion 12A to the third wiring portion 12C). The softening layer 14b may be the terminal portion (the first terminal portion 14A to the third terminal portion 14C) itself, may be formed by modifying the surface of the terminal portion, or may be formed by coating the surface with an organic conductive material such as conductive rubber, metal vapor deposition, or the like. According to the configuration, since the surface of the terminal portion is softened, the terminal portion can be deformed to follow the contact portion 21, so that the connection between the terminal portion and the contact portion 21 can be stabilized.

### Twentieth Embodiment

Next, a twentieth embodiment of the present invention will be described. In the following description, the same or equivalent configurations as those in the above-described embodiment are denoted by the same reference numerals, and descriptions thereof will be simplified or not be repeated.

FIG. 24 is a simplified view of the bioelectric potential measurement device 1 according to the twentieth embodiment. FIG. 24 is a schematic view corresponding to FIG. 12 described above.

As shown in FIG. 24, in the bioelectric potential measurement device 1 according to the twentieth embodiment, the contact portion 21 has a curved corner portion 21a. Specifically, the corner portion 21a where a lower end surface and a side surface of the contact portion 21 intersect with each other is formed in an arc shape in a longitudinal cross-sectional view along the up-down direction of the contact portion 21.

As described above, the contact portion 21 according to the twentieth embodiment has the curved corner portion 21a. According to the configuration, it is possible to reduce load (stress concentration) applied to the electrode sheet 10 from the corner portion 21a of the contact portion 21.

### Twenty-First Embodiment

Next, a twenty-first embodiment of the present invention will be described. In the following description, the same or equivalent configurations as those in the above-described embodiment are denoted by the same reference numerals, and descriptions thereof will be simplified or not be repeated.

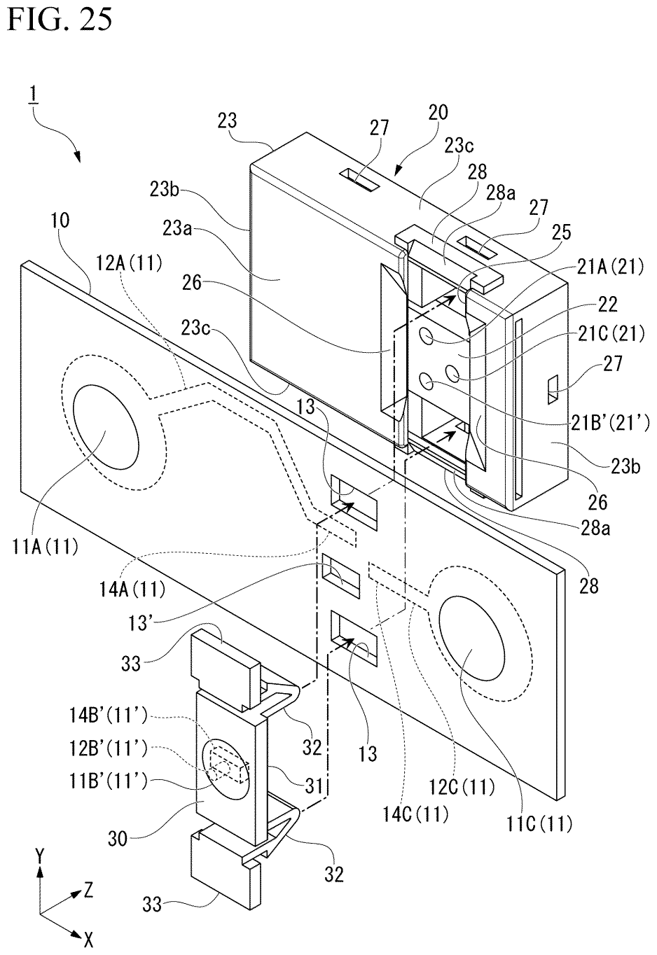

FIG. 25 is an exploded perspective view of the bioelectric potential measurement device 1 according to the twenty-first embodiment.

As shown in FIG. 25, in the bioelectric potential measurement device 1 according to the twenty-first embodiment, a connection member-side electrode 11′ which in contact with the biological side is provided on the connection member 30.

The connection member-side electrode 11′ shown in FIG. 25 includes an electrode portion 11B′ corresponding to the above-described second electrode portion 11B, a wiring portion 12B′ corresponding to the above-described second wiring portion 12B, and a terminal portion 14B′ corresponding to the above-described second terminal portion 14B. The electrode portion 11B′ is disposed on the biological side (−Z side) of the facing portion 31. The terminal portion 14B′ is disposed on the device side (+Z side) of the facing portion 31. The wiring portion 12B′ penetrates the facing portion 31 in the Z-axis direction, and connects the electrode portion 11B′ to the terminal portion 14B′.

The device 20 includes a second contact portion 21B′ which is connected to the connection member-side electrode 11′. A through-hole 13′ which brings the second contact portion 21B′ into contact with the terminal portion 14B′ is formed in the electrode sheet 10. According to the configuration, since the electrode portion 11B′ can be disposed at a position overlapping with the facing portion 31 of the connection member 30 in a plan view, a degree of freedom in the disposition of the electrode portion 11B′ can be improved.

### Twenty-Second Embodiment

Next, a twenty-second embodiment of the present invention will be described. In the following description, the same or equivalent configurations as those in the above-described embodiment are denoted by the same reference numerals, and descriptions thereof will be simplified or not be repeated.

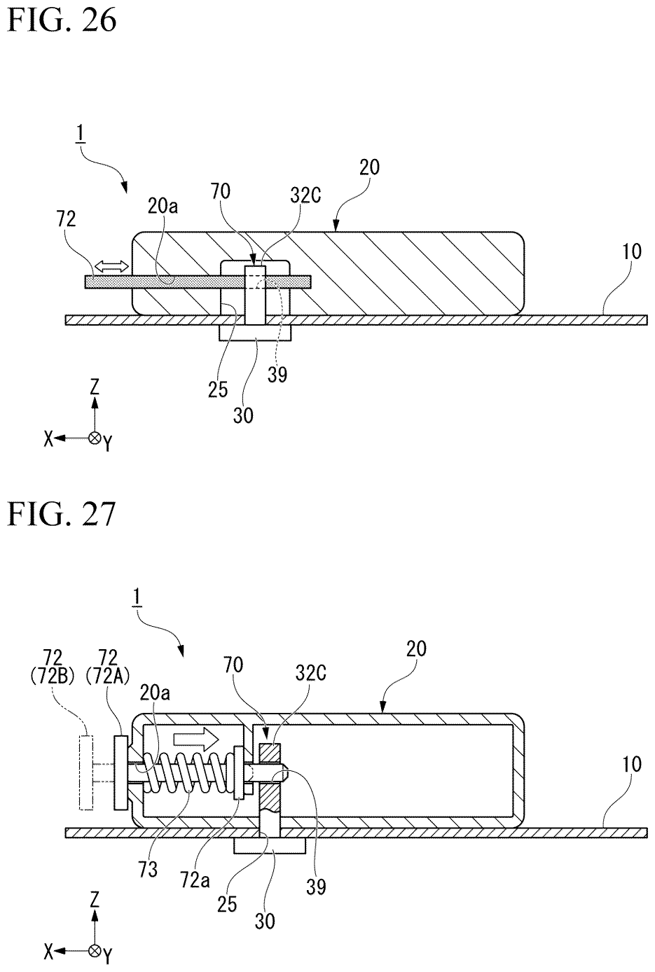

FIG. 26 is a schematic cross-sectional view of the bioelectric potential measurement device 1 according to the twenty-second embodiment along the longitudinal direction.

As shown in FIG. 26, the bioelectric potential measurement device 1 according to the twenty-second embodiment includes an attachment and detachment mechanism 70 in which the connection member 30 is attachable to and detachable from the device 20.

Specifically, the connection member 30 includes a fitting portion 32C in which a through-hole 39 penetrating in the X-axis direction is formed. The device 20 has an insertion hole 20a which is formed from a side surface of the device 20 on the +X side toward a space where the fitting portion 32C is disposed, the insertion hole 20a extending in the X-axis direction. The insertion hole 20a is formed by penetrating a space where the fitting portion 32C is disposed.

A rod-like movement member 72 which is movable in the X-axis direction is inserted into the insertion hole 20a. The movement member 72 is inserted into the through-hole 39 of the fitting portion 32C in the opening portion 25. According to the configuration, the connection member 30 can be easily attached to and detached from the device 20 by inserting and removing the movement member 72 from the device 20. Therefore, a force required to detach the electrode sheet 10 from the device 20 is less than that in a case where the fitting portion 32 having a claw shape as shown in FIG. 2 is elastically deformed, and thus the damage to the connection member 30 can be suppressed.

### Twenty-Third Embodiment

Next, a twenty-third embodiment of the present invention will be described. In the following description, the same or equivalent configurations as those in the above-described embodiment are denoted by the same reference numerals, and descriptions thereof will be simplified or not be repeated.

FIG. 27 is a schematic cross-sectional view of the bioelectric potential measurement device 1 according to the twenty-third embodiment along the longitudinal direction.

As shown in FIG. 27, the attachment and detachment mechanism 70 according to the twenty-third embodiment includes a movement member 72 which is movable between a fitting position 72A into which the fitting portion 32C fits and a non-fitting position 72B where the fitting portion 32C is disengaged from the fitting position 72A, and a biasing member 73 which biases the movement member 72 from the non-fitting position 72B toward the fitting position 72A.

Specifically, the movement member 72 includes a flange 72a which is in contact with the biasing member 73. The biasing member 73 biases the flange 72a toward the fitting portion 32C side (−X side) inside the device 20. As the biasing member 73, a coil spring can be mentioned; but a spring other than the coil spring may be used, or an elastic body such as rubber may be used.

According to the configuration, the movement member 72 fitted to the fitting portion 32C is moved from the fitting position 72A to the non-fitting position 72B against the biasing of the biasing member 73, so that the movement member 72 can be pulled out from the through-hole 39 of the fitting portion 32C. As a result, the connection member 30 can be easily detached from the device 20.

In a case where the connection member 30 is attached to the device 20, the fitting portion 32C is inserted into the opening portion 25 of the device 20 in a state in which the movement member 72 is pulled to be moved to the non-fitting position 72B. Thereafter, by releasing the hand from the movement member 72, the movement member 72 can be inserted into the through-hole 39 of the fitting portion 32C by a biasing force of the biasing member 73.

### Twenty-Fourth Embodiment

Next, a twenty-fourth embodiment of the present invention will be described. In the following description, the same or equivalent configurations as those in the above-described embodiment are denoted by the same reference numerals, and descriptions thereof will be simplified or not be repeated.

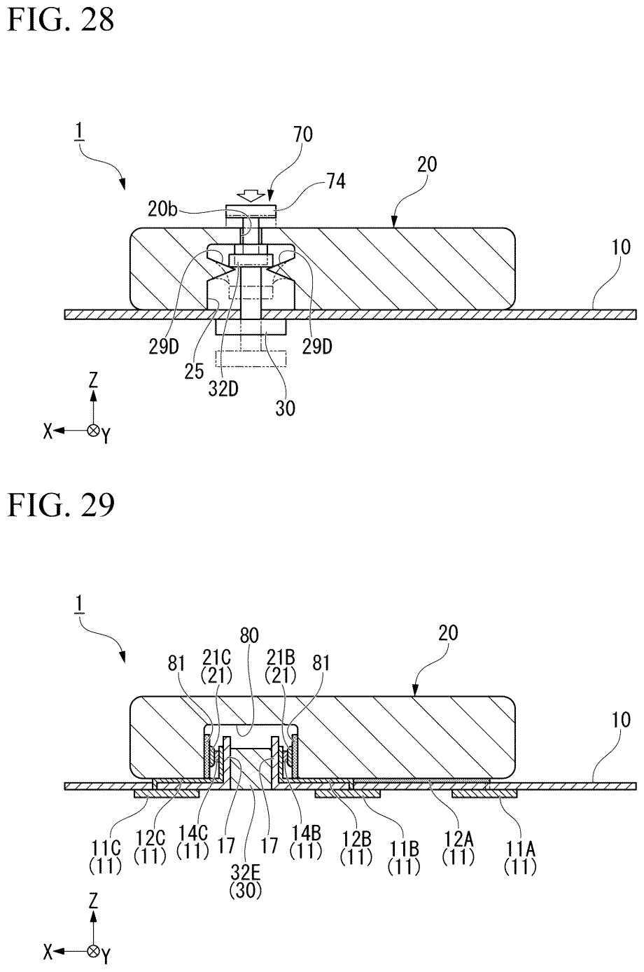

FIG. 28 is a schematic cross-sectional view of the bioelectric potential measurement device 1 according to the twenty-fourth embodiment along the longitudinal direction.

As shown in FIG. 28, the attachment and detachment mechanism 70 according to the twenty-fourth embodiment includes a button portion 74 which is displaced in response to attachment and detachment of the connection member 30.

Specifically, the button portion 74 is provided on an upper surface of the device 20. A lower end part of the button portion 74 extends to the inside of the opening portion 25 of the device 20, and faces a fitting portion 32D of the connection member 30 in the up-down direction (Z-axis direction). The fitting portion 32D fits into a fitting target portion 29D of the device 20. The fitting target portion 29D has an elastically deformable claw shape.

According to the configuration, in a case where the button portion 74 is pushed down, the fitting target portion 29D fitted to the fitting portion 32D is elastically deformed, and thus the fitting portion 32D can be moved downward from the fitting target portion 29D. As a result, the connection member 30 can be easily detached from the device 20 by displacing the button portion 74.

In addition, in a case where the connection member 30 is attached to the device 20, the fitting portion 32D can be inserted into the opening portion 25 of the device 20, and the fitting target portion 29D can be elastically deformed to move the fitting portion 32D upward from the fitting target portion 29D. In this case, since the button portion 74 is pushed up by the fitting portion 32D and is displaced upward, it is possible to check from the outside that the connection member 30 is in the fitted state.

### Twenty-Fifth Embodiment

Next, a twenty-fifth embodiment of the present invention will be described. In the following description, the same or equivalent configurations as those in the above-described embodiment are denoted by the same reference numerals, and descriptions thereof will be simplified or not be repeated.

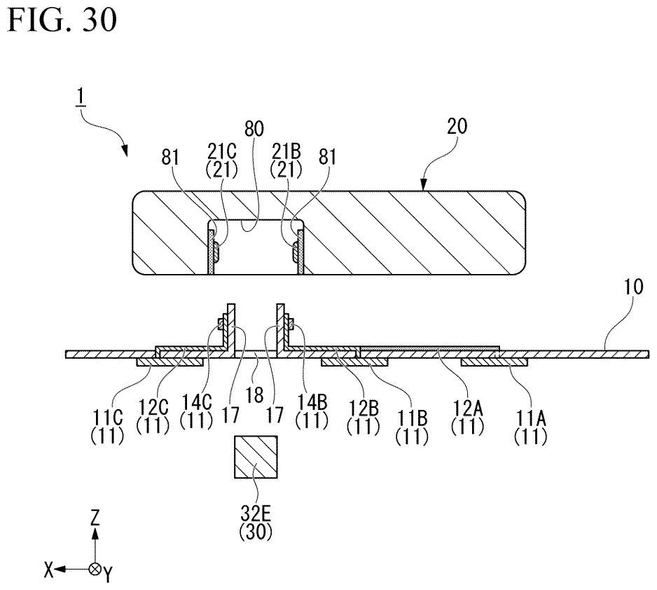

FIG. 29 is a schematic cross-sectional view of the bioelectric potential measurement device 1 according to the twenty-fifth embodiment along the longitudinal direction. FIG. 30 is an exploded view of the bioelectric potential measurement device 1 shown in FIG. 29.

As shown in FIGS. 29 and 30, the electrode sheet 10 according to the twenty-fifth embodiment has a bent portion 17.

As shown in FIG. 30, a notch portion 18 is formed in the electrode sheet 10. The notch portion 18 has, for example, an H-shape in a plan view or an S-shape in a plan view; and the bent portion 17 is formed by cutting out a part of the electrode sheet 10 to face in the X-axis direction. The bent portion 17 is provided with the terminal portion (the first terminal portion 14A (not shown), the second terminal portion 14B, and the third terminal portion 14C).

The device 20 has an accommodation portion 80. The accommodation portion 80 is open to the lower surface of the device 20. The contact portion 21 (the first contact portion 21A (not shown), the second contact portion 21B, and the third contact portion 21C) is provided in a portion of the side wall portion 81 of the accommodation portion 80, facing in the X-axis direction.

As shown in FIG. 29, the connection member 30 can be accommodated in the accommodation portion 80. The connection member 30 has a rectangular block-shaped fitting portion 32E which fits into the accommodation portion 80, and connects the terminal portion (the first terminal portion 14A (not shown), the second terminal portion 14B, and the third terminal portion 14C) to the contact portion 21 (the first contact portion 21A (not shown), the second contact portion 21B, and the third contact portion 21C).

As described above, in the twenty-fifth embodiment, the device 20 has the accommodation portion 80 in which the connection member 30 (fitting portion 32E) is accommodated; the accommodation portion 80 has the side wall portion 81 on which the contact portion 21 is disposed; the electrode sheet 10 has the bent portion 17 which is held between the side wall portion 81 and the fitting portion 32E in the accommodation portion 80; and the terminal portion to be connected to the contact portion 21 is provided on the bent portion 17. According to the configuration, since the connection member 30 does not need to be disposed on the electrode sheet 10 on the biological side (−Z side), the electrode sheet 10 is unlikely to be floated, and thus the peeling of the electrode sheet 10 from the biological side can be suppressed.

The contact portion 21 may be provided on a top wall surface of the accommodation portion 80 facing the −Z side, instead of the side wall portion 81 of the accommodation portion 80. Even in this case, the connection member 30 (fitting portion 32E) fits into the accommodation portion 80 in the same manner, and the contact portion 21 and the terminal portion can be connected to each other. According to the configuration, since the connection member 30 does not protrude to the biological side, it is possible to reduce discomfort on the biological side.

### Twenty-Sixth Embodiment

Next, a twenty-sixth embodiment of the present invention will be described. In the following description, the same or equivalent configurations as those in the above-described embodiment are denoted by the same reference numerals, and descriptions thereof will be simplified or not be repeated.

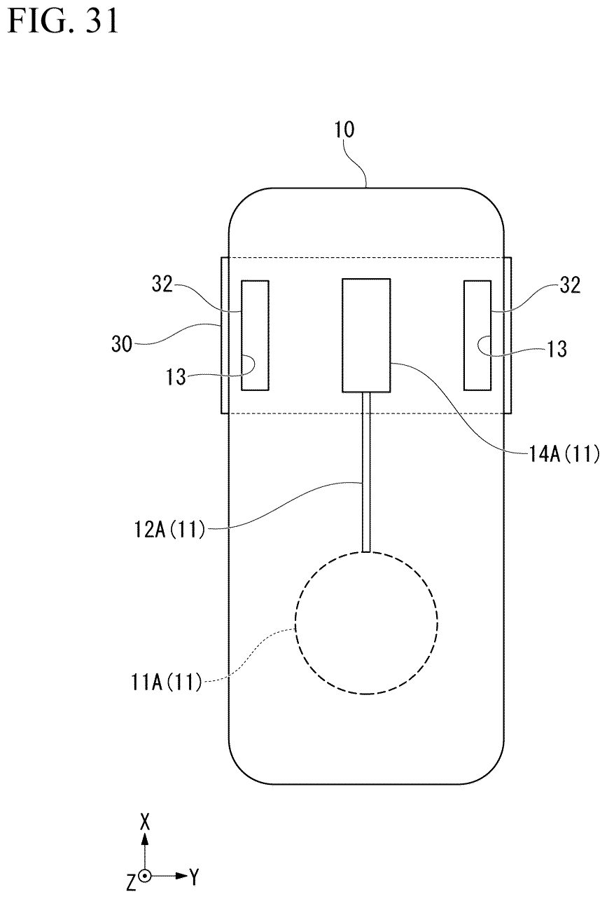

FIG. 31 is a plan view of the electrode sheet 10 according to the twenty-sixth embodiment.

As shown in FIG. 31, the electrode sheet 10 according to the twenty-sixth embodiment is integrated with the connection member 30.

The connection member 30 is integrated with the electrode sheet 10 with, for example, an adhesive. According to the configuration, the number of components of the bioelectric potential measurement device 1 is reduced, so that the bioelectric potential measurement device 1 is easily assembled. In addition, the fitting portion 32 of the connection member 30 is a restricting portion which restricts the deformation of the electrode sheet 10 in the first direction (Y-axis direction). According to the configuration, since the stretch and contraction of the electrode sheet 10 in the first direction is restricted, the connection between the first terminal portion 14A and the first contact portion 21A (not shown) can be stabilized. Although not shown in FIG. 31, the same effect can be obtained even with the second terminal portion 14B and the third terminal portion 14C.

### Twenty-Seventh Embodiment

Next, a twenty-seventh embodiment of the present invention will be described. In the following description, the same or equivalent configurations as those in the above-described embodiment are denoted by the same reference numerals, and descriptions thereof will be simplified or not be repeated.

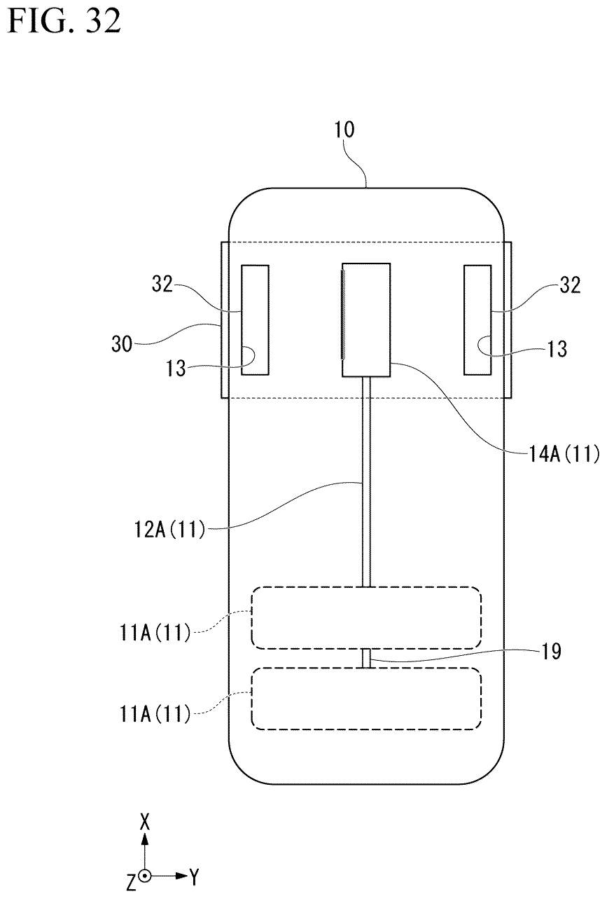

FIG. 32 is a plan view of the electrode sheet 10 according to the twenty-seventh embodiment.

As shown in FIG. 32, an electrode portion 11A according to the twenty-seventh embodiment is provided separately in the second direction (X-axis direction). According to the configuration, in a case where the electrode sheet 10 stretches and contracts in the second direction (X-axis direction), it is difficult for stress to be applied to the electrode portion 11A. The separated electrode portions 11A are connected to each other by a connection wiring 19.

### Twenty-Eighth Embodiment

Next, a twenty-eighth embodiment of the present invention will be described. In the following description, the same or equivalent configurations as those in the above-described embodiment are denoted by the same reference numerals, and descriptions thereof will be simplified or not be repeated.

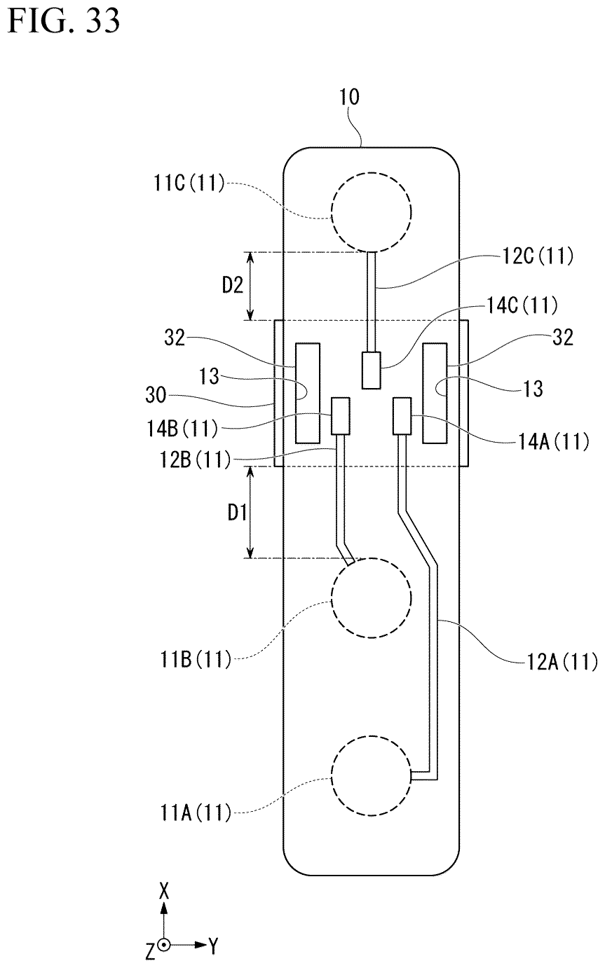

FIG. 33 is a plan view of the electrode sheet 10 according to the twenty-eighth embodiment.

As shown in FIG. 33, in the electrode sheet 10 according to the twenty-eighth embodiment, a second electrode portion 11B is disposed to be separated from the connection member 30 by a distance D1, and a third electrode portion 11C is disposed to be separated from the connection member 30 by a distance D2.

Specifically, the distance D1 is larger than, for example, a thickness of the facing portion 31 of the connection member 30 shown in FIG. 2 in the Z-axis direction. In addition, the distance D2 is also larger than the thickness of the facing portion 31 of the connection member 30 in the Z-axis direction. According to the configuration, the peeling of the second electrode portion 11B and the third electrode portion 11C due to the influence of floating of the electrode sheet 10 from the biological side, caused by the thickness of the facing portion 31, can be suppressed. In addition, since the first electrode portion 11A is disposed to be farther from the connection member 30 than the second electrode portion 11B, the influence of the floating caused by the thickness of the facing portion 31 is small.

### Twenty-Ninth Embodiment

Next, a twenty-ninth embodiment of the present invention will be described. In the following description, the same or equivalent configurations as those in the above-described embodiment are denoted by the same reference numerals, and descriptions thereof will be simplified or not be repeated.

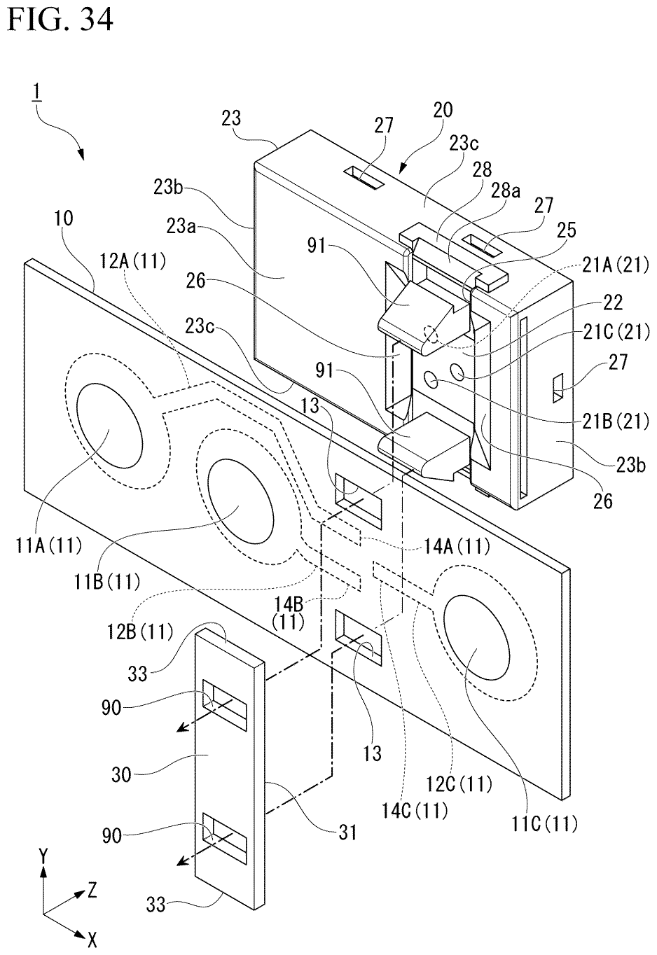

FIG. 34 is an exploded perspective view of the bioelectric potential measurement device 1 according to the twenty-ninth embodiment.

As shown in FIG. 34, in the bioelectric potential measurement device 1 according to the twenty-ninth embodiment, a fitting portion 91 is provided on the device 20 side, and a fitting target portion 90 is provided on the connection member 30.

Specifically, the fitting target portion 90 is a pair of through-holes formed in the facing portion 31 of the connection member 30. The fitting portion 91 is a pair of claws which can be inserted into and fitted to the fitting target portion 90, and is formed on the device 20. The pair of the fitting portions 91 pass through the pair of the through-holes 13 of the electrode sheet 10, and are fitted to the fitting target portion 90. Even with the configuration, the electrode sheet 10 can be connected to the contact portion 21.

While preferred embodiments of the present invention have been described and illustrated above, it should be understood that these are exemplary of the present invention and should not be considered as limiting. Additions, omissions, substitutions, and other changes can be made without departing from the scope of the present invention. Accordingly, the present invention should not be considered as being limited by the foregoing description, and is only limited by the scope of the appended claims.

In addition, for example, in the above-described embodiments, the configuration has been described in which the connection member 30 is integrated with the device 20 to be rotatable; but the connection member 30 may be integrated with the device 20 to be slidable.

What is claimed is:

1. A bioelectric potential measurement device comprising:
an electrode sheet configured to acquire a biological signal;
a device which has at least one contact portion to be connected to the electrode sheet; and
a connection member configured to connect the electrode sheet to the at least one contact portion by holding the electrode sheet between the device and the connection member,
wherein the connection member has at least one fitting portion which fits into the device, and
the electrode sheet has a shape corresponding to the at least one fitting portion.

2. The bioelectric potential measurement device according to claim 1,
wherein the device has a fitting target portion into which the at least one fitting portion to be fitted.

3. The bioelectric potential measurement device according to claim 1,
wherein the at least one fitting portion comprises a plurality of fitting portions, and
wherein the plurality of the fitting portions is provided in a pair in at least a lateral direction of the electrode sheet.

4. (canceled)

5. (canceled)

6. (canceled)

7. (canceled)

8. The bioelectric potential measurement device according to claim 1,
wherein the at least one contact portion comprises a plurality of contact portions, and
the connection member connects the electrode sheet to the plurality of the contact portions.

9. The bioelectric potential measurement device according to claim 1,
wherein a positioning mechanism which positions the electrode sheet and the at least one contact portion is provided, and
the positioning mechanism includes the at least one fitting portion.

10. The bioelectric potential measurement device according to claim 9,
wherein the positioning mechanism includes at least one through-hole which is formed in the electrode sheet corresponding to the at least one fitting portion and through which the at least one fitting portion is disposed.

11. The bioelectric potential measurement device according to claim 10,
wherein the at least one through-hole comprises a plurality of through-holes,
wherein the plurality of the through-holes is provided in a pair, and
the at least one contact portion is disposed between the pair of the through-holes in a plan view.

12. The bioelectric potential measurement device according to claim 9,
wherein the positioning mechanism includes a constricted portion which is formed on an outer edge of the electrode sheet corresponding to the at least one fitting portion and on which the at least one fitting portion is disposed.

13. (canceled)

14. (canceled)

15. (canceled)

16. (canceled)

17. (canceled)

18. (canceled)

19. The bioelectric potential measurement device according to claim 1,
wherein the connection member configured to be movable and integrated with the device.

20. (canceled)

21. (canceled)

22. (canceled)

23. (canceled)

24. The bioelectric potential measurement device according to claim 1,
wherein the electrode sheet has at least one conductive portion to be connected to the at least one contact portion, and
the at least one conductive portion includes a terminal portion which is in contact with the at least one contact portion, an electrode portion which is in contact with a biological side, and a wiring portion configured to connect the terminal portion and the electrode portion to each other.

25. (canceled)

26. (canceled)

27. The bioelectric potential measurement device according to claim 24,
wherein a surface of the terminal portion is harder than the wiring portion.

28. (canceled)

29. The bioelectric potential measurement device according to claim 24,
wherein the device has an accommodation portion in which the connection member is accommodated, and
the contact portion to which the terminal portion is connected is disposed in the accommodation portion.

30. (canceled)

31. The bioelectric potential measurement device according to claim 1,
wherein the at least one fitting portion has a restricting portion which restricts deformation of the electrode sheet in a first direction.

32. (canceled)

33. (canceled)

34. (canceled)

35. (canceled)

36. (canceled)

37. (canceled)

38. The bioelectric potential measurement device according to claim 1,
wherein the connection member is integrated with the electrode sheet.

39. The bioelectric potential measurement device according to claim 1,
wherein the device has an attachment and detachment mechanism which is attachable to and detachable from the connection member.

40. The bioelectric potential measurement device according to claim 39,
wherein the attachment and detachment mechanism includes a movement member which is movable between a fitting position where the fitting portion fits and a non-fitting position where the fitting portion is disengaged from the fitting position, and a biasing member which biases the movement member from the non-fitting position toward the fitting position.

41. (canceled)

42. The bioelectric potential measurement device according to claim 12,
wherein the at least one contact portion comprises a plurality of contact portions,
wherein the at least one constricted portion comprises a plurality of constricted portions formed on the outer edge of the electrode sheet and provided in a pair in a lateral direction of the electrode sheet, and
wherein the plurality of the contact portions are disposed between a pair of the constricted portions in a plan view of the electrode sheet.

43. The bioelectric potential measurement device according to claim 19,
wherein the connection member is integrated with the device as to be separable from the device.
